(19) United States
(12) Patent Application Publication
(10) Pub. No.: US 2004/0101934 A1
(43) Pub. Date: May 27, 2004

(54) Peptide derived from yeast having activities as anti-tsress, anti-fatigue and brain neurotrophic factor and relaxing premenstrual syndrome and menstrual pain, and preparing process thereof

(76) Inventors: Yun Seok Choe, Goyang-city (KR); Il Jun Kang, Chuncheon-city (KR); Hyung Joo Suh, Seoul (KR); Young Chun Choi, Chuncheon-city (KR); Hee Sun Yun, Seoul (KR); Kyung Mi Kim, Seoul (KR); Sang Wook Ahn, Seoul (KR)

Correspondence Address: CANTOR COLBURN, LLP, 55 GRIFFIN ROAD SOUTH, BLOOMFIELD, CT, 06002

(21) Appl. No.: 10/469,271

(22) PCT Filed: Feb. 27, 2002
(86) PCT No.: PCT/KR02/00324

(30) Foreign Application Priority Data
Feb. 27, 2001 (KR) 2001-0009946
May 14, 2001 (KR) 2001-0026208

(51) Int. Cl. C12P 19/34

(52) U.S. Cl. 435/91.1

(57) ABSTRACT

A yeast-derived bioactive peptide having activities as an anti-stress agent, an anti-fatigue agent, premenstrual syndrome(PMS) and menstrual pain relaxants, and a brain-neurotrophic factor, and a method for preparing the bioactive peptide are provided. The preparation method involves incubating a strain of yeast, which is a natural source reconigzed as safe, until a maximum growth phase. The generation and release of anti-stress substances beneficial to the human body from the yeast are induced by properly applying a physical or chemical stress, such as high-temperature heating, ultrasonic waves, vibration, pH variations, etc. Next the yeast is autolyzed and purified to obtain the bioactive peptide. The yeast-derived bioactive peptide is effective in relieving stress, nervousness, anxiety, tension, insomnia, fatigue, and imbalance in the autonomic nerve regulation. Therefore, the yeast-derived bioactive peptide is available as an anti-stress agent, an anti-fatigue agent, premenstrual syndrome and menstrual pain relaxants, a hain-neurotrophin, and a source of active foods having these activities.

### TECHNICAL FIELD

The present invention relates to a yeast-derived bioactive peptide having activities as an anti-stress agent, an anti-fatigue agent, premenstrual syndrome and menstrual pain relaxant, and a brain-neurotrophic factor, and a method for preparing the bioactive peptide, and more particularly, to an bioactive peptide obtained by hydrolyzing and purifying yeast approved as a food-grade, protein-based active material and having activities as an anti-stress agent, an anti-fatigue agent, premenstrual syndrome and menstrual pain relaxants, and a brain-neuroprophic factor.

### BACKGROUND ART

Modern peoples suffer from various types of daily stress. Emotional changes caused by such stress affect the autonomic nervous system, hormone secretion, and immune system in the human body and further ones' overall heath.

In general, when the human body is stressed, due to stimulation of the sympathetic part of the autonomic nervous system, secretion of hormones, in particular, adrenaline, is triggered (Corrodi H, Fuxe K, Hokfelt T.; The effect of immobilization stress on the activity of central monoaminergic neuron, Life Science 7: 108-112, 1968). To inhibit continuous stimulation of the sympathetic part, the parasympathetic part is spontaneously stimulated to secrete acetylcholine, and thus the body maintains balance in the autonomic nervous system. However, when the human body is subjected to excess stress for a long period of time, the body is too exhausted to effectively manage the stress accumulated in the sympathetic part, so balance in the autonomic nervous system is destroyed and directly affect the mechanisms of cellular and humoral immunity, thereby causing immunodeficiency, functional disorders. This abnormal state if it is prolonged causes organ disorders.

As a result, peptic ulcer, hypertension, cancer, diabetes, irritable colon syndrome, cardiopathy, bronchial asthma, tension headache, arthritis, neurodermatitis, etc. may result. Typical symptoms include liability to fatigue, impatience in daily life, inability to fall into a deep sleep, chills, sweating, shoulder pains, oppressed feeling, feeling as if something is in throat, dizziness, hyposexuality for males, and infertility for females.

To alleviate those stress disorder, conventionally, psychotropics have been used; for example, minor tranquillizers such as diazepam, meprobamate, methylpentinol, and etifoxine; neuroleptics such as chloropromazin, promethazine, and azapaerone; beta-adrenergic antagonist such as bunitrol; antidepressants such as a triple- or quadruple-ring compound, which are used alone or together with a neuropleptic; psycoanleptics such as caffeine, amphetamine, or derivatives thereof; and sedatives and hypnotics such as a phenobarbital-codein complex (Poldinger, W., Schmidlin, P. E., Wider, F., Index Psychopharmacorum, H. Buber, Bern). However, the use of those psychotropics relies on pharmacotherapy for relief of a predominant symptom without pathological consideration of the cause of the stress and cannot reduce chromatic damages caused from the stress. Also, the psychotropics cannot resist the stress through catabolic regeneration but rather inhibits a normal reaction in that body is adapted to stress and causes a number of adverse effects. Typically, Human body develops tolerance to the antianxiety agents and psychoanaleptics such as amphetamine or caffeine and thus dosage of above drugs need to be increased. A significant adverse effect is that there is the probability of becoming dependent on the psychoactive agent.

To eliminate such imbalance in the autonomic nervous system due to stress, many attempts have been made in a variety of aspects. In particular, stress is medically defined as a negative stimulus destructing the body's homeostasis. Neurotransmitters are involved in the negative stimulus. Acetylcholine derived from cholesterol, described above, is an important neurotransmitter. Acetylcholine, a relaxation-inducing neurotransmitter, is secreted from the parasympathetic part of the autonomic nervous system. About 50 other neurotransmitters have been discovered so far.

Such a neurotransmitter needs a complementary counterpart called “receptor” for it to function. Although a number of neurotransmitters exist, the neurotransmitters cannot function properly if there is no receptor having a peptide structure to be coupled to the neurotransmitter. A muscarinic receptor, which is coupled to acetylcholine, is composed of peptides including aspartic and glutamic residues, which are important for the coupling, and hydrophobic amino acids surrounding the residues (Gearien 1999). When a neurotrophic factor including these peptides is supplied to the body, nerve cells are nourished and grown to treat a variety of neuropathies, such as Parkinson's disease, without side effects. By accelerating the generation of sufficient neurotransmitters and their receptors, impulses on nervous system by excess stress (stimuli) can be absorbed and delivered without causing a load to the nerve cells, thereby treating stress disorders. In other words, to intensify the nervous system, it is important to take in peptides acting as a neurotrophic factor to generate neurotransmitter receptors as well as neurotransmitters themselves. Since receptor peptides react depending on the amount of neurotransmitters, there is no side effect due to the excess dose of the receptor peptide.

A “brain-neurotrophic factor” refers to a neurotrophin for nerve tissues, such as the brain and spinal marrow, to accelerate the growth of the nerve cells or neuroglia cells. In th past, it was believed that brain nerve cells could not be grown. However, since then it has been discovered that brain nerve cells grow and proliferate with the supply of a particular neurotrophic factor and interest in studying neurotrophins has been increased. Accordingly, neurotrophins to regulate the growth and proliferation of nerve cells and their peptide sequences have been discovered with fetal and animal brains. Neurotrophins having peptides capable of accelerating the growth and proliferation of the nerve cells or neuroglia cells have been known to be effective for the treatment of functional disorders caused from nerve cell degeneration, such as Parkinson's disease and Alzheimer's disease (Varon and Bunge 1997, Ann. Rev. Neuroscicence 1:327; Thoenen and Edgar 1985, Science 229:238).

Significant neurotrophic factors found to date, include nerve growth factor (NGF), brain-derived neurotrophic factor (BDNF), glial cell-derived neurotrophic factor (GDNF), Neurotrophin-3 (NT-3), NT4/5, etc., which are kinds of peptides. However, use of a human or fetal brain to find a new neurotrophic factor raises ethical issues, because it is extracted from a corpus, and the amount of neurotrophic factor is too trace to be detected in the brain. So, only a few neurotrophic factors have been found up to now. Therefore, many kinds of neurotrophic factors, more than those identified to date, are predicted to exist.

Conventionally, a neurotrophic factor or neurotransmitter has been obtained by preparing a synthetic peptide or recombinant peptide using a peptide synthesizer or a genetic recombinant technique through polymerization chain reaction (PCR), respectively. However, these techniques are costly and cause a safety problem, thereby limiting commercial applications. Most synthetic or recombinant peptides are macromolecules of 30,000 daltons or greater, so they cannot pass through a brain blood barrier and reach a brain nerve cell through a brain blood barrier by oral administration, and thus direct injection into the brain is necessary (Medical Report 1998, Editions of Jan. and Feb.).

Therefore, it would be desirable to prepare a neurotropic peptide, using a yeast extract or yeast peptide derived from a food-grade yeast as in the present invention, having anti-stress, anti-fatigue, anti-anxiety, and deep sleep-inducing effects with a comparatively small dose, without using complicated processes of the genetic recombination method. This neurotropic peptide derived from the yeast according to the present invention can be widely applied for commercial use, compared to conventional neurotropic factors (Neurotrphin, NT-3, BDNF, NGF, etc.) identified by genetic recombination and does not cause a safety problem, such as suspicion of a genetic mutant.

Yeast, generally recognized as safe (GRAS) for the human body, contains 50% or more quality proteins, excess minerals, vitamin B, etc., so it has been widely used in the liquor or bakery industry as a source of protein, nucleic acids, enzymes, liquids, vitamins, minerals, etc. (Roman et al., Food Biotechnology, 6, 225, 1992). Yeast extracts produced by autolytic enzyme or other proteases have been used as a source of microorganism fermentation media, seasonings, and health foods (Bioindustry, 14, 53, 1997). However, the functionality of the yeast extract hydrolyzed from yeast or yeast-derived peptides and their specific use as an anti-stress agent and a native brain-neurotrophic factor through experimental assays have not been reported yet. Also, the production of anti-stress and anti-anxiety agents, sleeping drugs, and other medicines using the yeast-derived peptides has not been disclosed. In addition, the effect of the yeast extract on premenstrual syndromes similar to stress symptoms or on menstrual pains is not known.

Premenstrual syndromes (PMS) refer to symptoms experienced by fertile women during their menstrual cycle after ovulation, including physical symptoms such as cramp pain, low back pain, peycalgia, abdominal bloating, diarrhea, constipation, and breast fullness and tenderness, and emotional symptoms such as anxiety, irritability, depression, insomnia, fatigue, reduction in concentration, idioctonia impulse, etc. Those symptoms are similar to stress symptoms and are experienced by 70% of all women, unendurable to 20% of those women, thereby causing social and economical losses due to the inability to work.

Menstrual pains refer to mild or incapacitating cramp pains or low back pain generally experienced by most women, about 50% of all fertile women, combined with PMS before, after, or during their menstrual cycle. Mostly, young women within 1 or 2 years after their menarche suffer from menstrual pains, but this may be sustained into their forties. Reportedly, about 10% of those feels so painful not to able to ordinary work for 1 to 3 days a month. According to the result of a survey by Kyunghee University Oriental Medicine Hospital, 47% suffered from menstrual cramp pain, low back pain, and more seriously, headaches, during their menstrual cycle, 13% experienced disturbance gastrointestinal such as anorexia and indigestion, and 8% had disesthesia. In particular, about 70% of 632 middle and high school girls, 50% of those seriously, experienced menstrual pains. Such serious symptoms for the students are believed to be due to stress from excess schoolwork and examination. It was also investigated that above 90% of those basically do not manage their pain, 66% of those endure the pain without any treatment, and 28% of those take analgesics.

In spite of the efforts made by many researchers over a long period of time, the causes of PMS or menstrual pains have not been accurately identified yet. The relevancy of incretory hormonal imbalance during menstrual cycle to PMS or menstrual pains has been perceived.

There are several pathogeneses for PMS and menstrual pains. The pathogeneses may include considering those arising from the deficiency of progesterone, the excess secretion of estrogen and androgen, the excess secretion of pain-inducing prostaglandin, or the deficiency of vitamin B complex or essential fatty acids. In general, it is believed that PMS or menstrual pains occur due to the combination of the above-listed factors together with an environmental factor.

According to the most convincing pathogenesis for PMS and menstrual pains related with the excess secretion of prostaglandin, in the female menstrual cycle, as the secretion of a luteinizing hormone (LH), progesterone, stops, the pain inducing prostaglandin is secreted. As a result, tunica myometrium is contracted, and transient anemia and menstrual pains occur. According to this theory, to suppress such PMS or pains, the following methods have been suggested.

As a most widely used method, the production of prostaglandin is suppressed with the administration of, for example, aspirin or ibuprofen to relieve the menstrual pains. In another method, an anti-anxiety agent or anti-depressant, such as benzodiazepin, is used. Alternatively, progesterone is administered in the luteinizing phase of the female menstrual cycle.

In another treatment method, Korean Patent No. 0171408 discloses the use of melatonin (N-aceryl-5-methoxytriptamine), and Korean Laid-open Application No. 2001-0024462 discloses the use of cerotonin (5-hydroxytriptamine).

Among a number of methods for treatment of PMS or menstrual pains, treatments with hormones, such as progesterone, melatonin, or cerotonin, or with neurotransmitters in the luteinizing phase are known to be effective for relieving general pains, stress, chronic fatigue, and depression as well as PMS or menstrual pains.

However, those treatment methods of direct administration of hormones, such as melatonin or cerotonin, cannot ensure 100% safety, and the cost of preparing the pharmaceuticals is high. Therefore, the treatment agents are not generally taken.

Yeast is known to respond sensitively to external conditions, compared to other microorganisms. Yeast has the ability to grow in both anaerobic and aerobic conditions, stops growing if the condition of a growth medium is unsuitable for growth, and undergoes heterozygosis to sustain itself under poor external environments. All organisms exhibit an alarm reaction when a stress is perceived for the first time and actively resists against the stress if the stress is not relieved to induce physiological changes for homeostasis (Seyle, 1956). The inventor has realized the present invention by combining the above characteristics of yeast and organisms. In particular, after full proliferation of yeast, the growth medium was subjected to physical and chemical stresses, such as high-temperature heating, ultrasonic waves or vibrations, and pH variations, of a degree not to causing destruction, to produce excess anti-stress substances such as proteins and enzymes. A yeast extract was prepared by autolysis or hydrolysis with a protease, and purified by ultrafiltration to attain yeast-derived peptides. Also, it was proven that the yeast extract and peptides have activities as brain-neurotrophins (derived from the natural source), therapeutic and prophylactic agents for the treatment of autonomic nerve disorders, such as an anti-stress agent, anti-anxiety agent, or sleeping aids, and PMS and menstrual pain relaxants, the PMS and menstrual pains showing similar symptoms to stress.

### DISCLOSURE OF THE INVENTION

It is an object of the present invention to provide a new yeast extract and bioactive peptides obtained by hydrolyzing yeast, which have activities as an anti-stress agent, an anti-fatigue agent, a natural brain-neurotrophin, and premenstrual syndrome and menstrual pain relaxants.

It is another object of the present invention to provide a natural complex neurotrophin containing a yeast extract or yeast-derived peptide as an active component to treat stress disorders and maintain balance in the human autonomic nervous system.

It is another object of the present invention to provide an anti-stress agent containing a yeast extract or yeast-derived peptide as an active component having activities as an anti-stress agent, an anti-anxiety agent, a tranquilizer, and a sleeping aids to treat neurogenic disorder, especially imbalance in the autonomic nervous system, caused from stress.

It is another object of the present invention to provide an anti-fatigue agent containing a yeast extract or yeast-derived peptide as an active component having the activity to recovery fatigue caused from stress or muscular fatigue caused from exercises.

It is another object of the present invention to provide an natural supplement containing a yeast extract or yeast-derived peptide as an active component having the activity to reduce premenstrual syndromes (PMS) and menstrual pains similar to symptoms from stress and chronic fatigue.

It is another object of the present invention to provide an active beverage containing an anti-stress substance extracted from a natural source proven to be safe, not from a synthetic medicine having serious side effects, which can be conveniently and safely taken to relax stress.

To achieve an object of the present invention, there is provided a yeast extract derived from yeast and having activities as an anti-stress agent, an anti-fatigue agent, premenstrual syndrome and menstrual pain relaxants, and a neurotrophic factor.

In the yeast extract according to the present invention, the source yeast contains excess high-quality proteins, minerals, and B vitamins. The yeast extract is obtained by known general extractions methods, for example, using an autolytic enzyme or protease. Commercially available yeast extracts were found to have activities as an anti-stress agent, an anti-stress agent, PMS and menstrual pain relaxants, and a brain-neurotrophic factor, like the yeast extract according to the present invention.

The yeast extract according to the preset invention having activities as an anti-stress agent, an anti-fatigue agent, premenstrual syndrome and menstrual pain relaxants, and a neurotrophic factor, is prepared by autolyzing the yeast preferably at a temperature of about 35-70° C., and more preferably, a temperature of about 50-60° C.

Only with the autolysis at a high temperature greater than or equal to about 35° C., an effective anti-stress activity can be induced to the yeast extract. It is believed that heating the yeast simultaneously triggers the release of stress-resistant substances to provide an anti-stress effect during autolysis. Alternatively, by applying an additional stress such as ultrasonic waves or vibrations, a new strain of yeast with enhanced stress resistance can be screened. The anti-stress activity of the yeast extract according to the present invention can be further improved by repeatedly applying such stresses to enhance the release of stress-resistant substances. In the yeast extract according to the preset invention having activities as an anti-stress agent, an anti-fatigue agent, premenstrual syndrome and menstrual pain relaxants, and a neurotrophic factor, it is preferable that the yeast is hydrolyzed with a protease during or after the autolysis. According to this invention, only a supernatant obtained by centrifuging hydrolytes produced in the hydrolysis may be included in the yeast extract according to the present invention having activities as an anti-stress agent, an anti-fatigue agent, premenstrual syndrome and menstrual pain relaxants, and a neurotrophic factor.

Also, the present invention provides a yeast-derived peptide having activities as an anti-stress agent, an anti-fatigue agent, premenstrual syndrome and menstrual pain relaxants, and a neurotrophic factor, the yeast-derived peptide characterized by including molecules of 10,000 daltons or less obtained by ultrafiltrating the supernatant from the centrifugation performed in the above preparation of the yeast extract according to the present invention.

In the yeast-derived peptide according to the present invention, the molecular weight cutoff value of 10,000 was determined by considering a variety of activities of peptides as an active food source to regulate body functions (New Technology Trend Report of 2000, Active Food, Korean Industrial Property Office). In general, molecules of a molecular weight of 10,000 or less are called “peptides”. The brain has a brain blood barrier which blocks macromolecules of a molecular weight of 15,000 or greater to protect the cerebrovascular system. Therefore, the yeast-derived peptide according to the present invention having a molecular weight of 10,000 or less can easily pass through the brain blood barrier.

Alternatively, a yeast-derived peptide according to the present invention having activities as an anti-stress agent, an anti-fatigue agent, premenstrual syndrome and menstrual pain relaxants, and a neurotrophic factor is characterized by comprising glutamic acid and aspartic acid of about 15-25 mol % each.

Since the yeast-derived peptide according to the present invention is rich in glutamic acid and aspartic acid, which are amino acids involved in the synthesis of muscarinic acetylcholine receptors, it is effective in alleviating stress disorders caused by the imbalance in the autonomic nervous system, including PMS and menstrual pains.

To achieve another object of the present invention, there is provided a method for preparing a yeast extract having activities as an anti-stress agent, an anti-fatigue agent, premenstrual syndrome and menstrual pain relaxants, and a neurotrophic factor, the method comprising: incubating a strain of yeast until a maximum growth phase; inducing autolysis of the yeast at a temperature of about 35-70° C. and simultaneously hydrolyzing the yeast with an addition of a protease; and attaining a supernatant by centrifuging the hydrolytes from the yeast.

Alternatively, the method for preparing the yeast extract according to the present invention may further comprise screening a new strain of the yeast survived resisting to stress by heating at high-temperature, applying ultrasonic waves and vibrations, and changing pH to a degree not to cause destruction before the incubation until the maximum proliferation stage.

Alternatively, the method for preparing the yeast extract according to the present invention may further comprise inducing secretion of metabolites resistant to stress by applying a physical or chemical stress selected from the group consisting of high-temperature heating, ultrasonic waves, vibrations, and pH variations, to a degree not to cause destruction before the autolysis.

This is based on the fact all organisms exhibit an alarm reaction when a stress is perceived for the first time and actively resists against the stress if the stress is not relieved to induce physiological changes for homeostasis (Seyle, 1956). In other words, when yeast is subjected to stress, it secretes a variety of enzymes to resist against the stress. The released enzymes are low molecular weight proteins having an activity as a neurotrophic factor to relieve the human body of the stress. Based on this fact, the yeast extract preparation method according to the present invention has been realized.

Physical or chemical stimuli applied to yeast in the preparation of a yeast extract according to the prevent invention to induce the generation and release of anti-stress substances from the yeast include heating at a temperature of about 35-45° C. which is higher than the optimum growth temperature of the yeast, ultrasonic waves, vibrations, and pH variations, to a degree not to destroying the yeast.

Preferably, the method for preparing the yeast extract according to the present invention comprises: screening a new strain of yeast resistant to stress by incubating the yeast with applications of ultrasonic waves and vibrations; inoculating the screened strain of the yeast on YM medium, incubating the medium at a temperature of about 22-25° C. until its exponential growth phase, collecting the yeast cells by centrifugation, and diluting the collected cells with a 1%-peptone buffer; applying ultrasonic waves and vibrations to the dilute at a high temperature of about 22-25° C. as stresses for 8 hours to induce the generation and release of stress-resistant metabolites from the yeast; autolysing the yeast product at a temperature of about 50-60 22-25° C. and simultaneously hydrolyzing the yeast product with the addition of a protease; and centrifuging hydrolytes to obtain a supernatant.

To achieve another object of the present invention, there is provided a method for preparing a yeast-derived peptide having activities as an anti-stress agent, an anti-fatigue agent, premenstrual syndrome and menstrual pain relaxants, and a neurotrophic factor, the method characterized by comprising obtaining peptides only having a molecular weight of 10,000 daltons or less by ultrafiltrating the supernatant from the centrifugation in the preparation of the yeast extract according to the prevent invention.

According to the present invention, only low molecular weight yeast-derived peptides of 10,000 or less is selected by ultrafiltrating the supernatant from the centrifugation described above through a membrane having a molecular weight cutoff value of 10,000. As a result, substances that would drop the efficacy of the yeast extract are removed while the low molecular weight substances capable of easily being absorbed into the body and capable of passing through the brain blood barrier with high bioavailability are separated and purified.

Alternatively, the method for preparing the yeast extract or the yeast-derived peptide according to the present invention may further involve drying and grinding the yeast extract or the yeast-derive peptide.

To achieve another object of the present invention, there is provided an anti-stress agent comprising the yeast extract or the yeast-derived peptide prepared by any of the above-described methods according to the present invention as an active component.

The anti-stress agent according to the present invention has an activity as a tranquilizer, a relaxant, an anti-anxiety agent, or a sleeping aids, and is effective in alleviating PMS and menstrual pains.

To achieve another object of the present invention, there is provided an anti-fatigue agent (fatigue recovering agent) comprising the yeast extract or the yeast-derived peptide prepared by any of the above-described methods according to the present invention as an active component.

To achieve another object of the present invention, there is provided a neurotrophin (neurotrophic factor) comprising the yeast extract or the yeast-derived peptide prepared by any of the above-described methods according to the present invention as an active component.

The present invention also provide a PMS and menstrual pain relaxant composition comprising the yeast extract or the yeast-derived peptide prepared by any of the above-described methods according to the present invention as an active component.

Preferably, the PMS and menstrual pain relaxant composition according to the present invention comprises: about 10-90% by weight dried powder of the yeast extract or the yeast-derived peptide; about 5-80% by weight chitosan; and about 5-80% by weight herbal powder of 5-80% by weight, based on the total weight of the premenstrual syndrome and menstrual pain relaxant composition. In this case, the citosan may be a water-soluble macromolecule of a molecular weight greater than or equal to about 300,000. Preferably, the herbal powder is derived from at least one selected from the group consisting of Korean angelica root, Salviae Radix, Curcuma aromatica, Zedoariae Rhizoma, mint, liquorice, ginger, gastrodia, white atractylis, Cnidium officinale, cinnamon, and ginseng.

To achieve another object of the present invention, there is provided an active beverage having activities as an anti-stress agent, an anti-fatigue agent, premenstrual syndrome and menstrual pain relaxants, and a neurotrophic factor, the active beverage comprising the yeast extract or the yeast-derived peptide prepared by any of the above-described methods according to the present invention.

The yeast extract or yeast-derived peptide contained in the active beverage according to the present invention contains anti-stress substances released by applying stress to yeast recognized as safe. Therefore, the active beverage prepared using the yeast extract or yeast-derive peptide according to the present invention provides an anti-stress effect as a tranquilizer, a relaxant, a sleeping drug, etc., and can be conveniently taken without concern about any side effect.

Preferably, the active beverage according to the present invention comprises: about 0.1-10% by weight the yeast extract or the yeast-derived peptide by any of the above-described methods according to the present invention; about 10-25% by weight common additives for beverage including a sweeter and an acidulant, based on the total weight of the bioactive beverage; and the balance water.

In the present invention, the common additives for beverage include liquid fructose, sucrose, maltodextrin, glucose, citric acid, nicotinamide, pantothenic acid, sodium benzoate, and kinds of flavors. Preferably, in the preparation of the bioactive beverage according to the present invention, any fruit juice, for example, ume juice, is added.

The present invention also provides a method for preparing an bioactive beverage having activities as an anti-stress agent, an anti-fatigue agent, premenstrual syndrome and menstrual pain relaxants, and a neurotrophic factor, the method comprising: incubating a strain of yeast until a maximum growth phase; inducing autolysis of the yeast at a temperature of about 35-70° C. and simultaneously hydrolyzing the yeast with an addition of a protease; attaining a supernatant by centrifuging hydrolytes produced in the hydrolysis; mixing the supernatant with active carbon in water and sterilizing the mixture under pressure; and purifying the sterilized mixture until it loses its color by filtering the sterilized mixture with suction.

In preparing a beverage with the yeast extract containing anti-stress substances, purification is necessary to remove the yeast odor. For the purification, after mixing the prepared yeast extract with active carbon and water, the mixture is sterilized under pressure and filtered. Active carbon almost not adsorbs amino acid (Hyung-ik Song and Jung-yeub Shin, Contemporary Fermentation Engineering, 1998, 293). Based on this nature of active carbon, the yeast extract was mixed with active carbon for decolorization and deodorization in the present invention.

The active beverage according to the present invention having activities as an anti-stress agent, an anti-fatigue agent, premenstrual syndrome and menstrual pain relaxants, and a neurotrophic factor, is prepared by diluting the beverage concentrate after the filtering. An bioactive beverage according to the present invention can be prepared by any general beverage preparation method without limitation, as long as the yeast extract prepared by any of the methods described above according to the present invention is incorporated therein.

Preferably, the active beverage preparation method according to the present invention further comprises: screening a new strain of yeast survived resisting to stress by heating at high-temperature, applying ultrasonic waves and vibrations, and changing pH during incubation to a degree not to causing destruction; and inducing secretion of metabolites resistant to stress by applying a physical or chemical stress selected from the group consisting of high-temperature heating, ultrasonic waves, vibrations, and pH variations, to a degree not to causing destruction.

In the preparation of the bioactive beverage according to the present invention, it is preferable that the autolysis is performed at a temperature of about 50-60° C., and the pressure sterilization is performed at about 1.5 atm and a temperature of about 100-125° C. for about 10-15 minutes.

### BRIEF DESCRIPTION OF THE DRAWINGS

FIG. 1 is an illustration of a muscarinic acetylcholine receptor map;

FIG. 2 is a graph of the result of Anti-stress Activity Test (1) for the present invention;

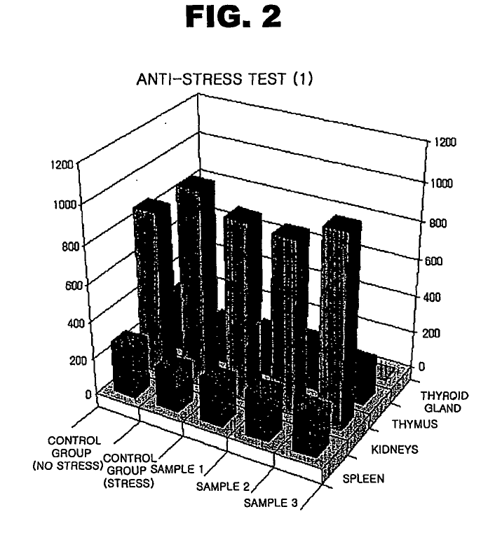

FIG. 3 is a graph of the result of Anti-stress Activity Test (2) for the present invention;

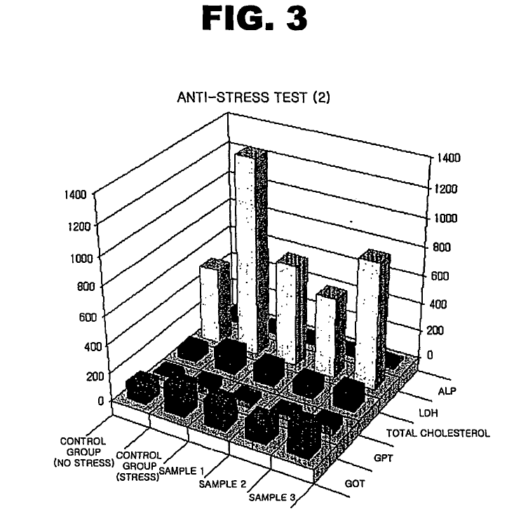

FIG. 4 shows an experimental swimming pool used to measure mouse' swimming endurance as a measure of anti-fatigue effects;

FIG. 5 is a graph of the result of an anti-fatigue activity test for the present invention;

FIGS. 6A and 6B are brain maps obtained as a result of a clinical test for the anti-stress effect of the present invention;

FIG. 7 is a gel filtration chromatograph illustrating the molecular weight distribution of yeast-derived peptides according to the present invention; and

FIG. 8 is a graph showing the degree of bone marrow cell proliferation with respect to different doses of the yeast-derived peptide according to the present invention; and

FIG. 9 is a graph showing the relative activity of a macrophage lysosomal enzyme with respect to different doses of the yeast-derived peptide according to the present invention.

### BEST MODE FOR CARRYING OUT THE INVENTION

Hereinafter, the present invention will be described in greater detail.

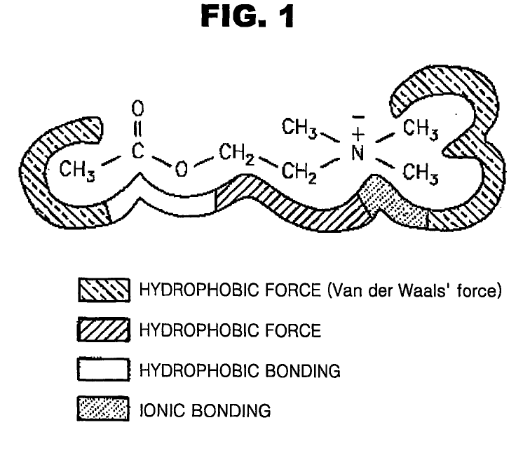

To recover the human organs subjected to sustained stimulation of the sympathetic part due to stress, which causes imbalance in the autonomic nervous system, the parasympathetic part needs to be actively stimulated. To this end, there is a need to accelerate release of the neurotransmitter acetylcholine at synapses. In addition, acetylcholine receptors are necessary to efficiently deliver the released acetylcholine to neurons. Acetylcholine is bound to muscarinic receptors, as shown in FIG. 1. The presence of more muscarinic receptors in the synapsis results in more efficient transmission of acetylcholine. Therefore, more muscarinic receptors need to be synthesized. The muscarinic receptors are composed of seven fractions of protein and are believed to have a negatively charged site to couple with quaternary ammonium salt of acetylcholine significant for the muscarinic activity. The negatively charged site is considered to be derived from acidic amino acid residues, such as aspartic acid and glutamic acid (Keun-il Kang, 1992, Introduction to Medicinal Chemistry) Therefore, peptide drugs enriched with acidic amino acids, aspartic acid and glutamic acid, to be used as a source material for the muscarinic acetylcholine receptors facilitates the biosynthesis of the muscarinic receptors in the human body and affects efficient transmission of acethylcholine of the parasympathetic part, thereby recover the imbalance in the autonomic nervous system due to the excess stimulation of the sympathetic part. As a result, the pathological conditions, both physical and emotional, caused from stress can be fundamentally inhibited or relieved.

The inventor has found that yeast extract is rich in amino acids effective in synthesizing the muscarinic receptors described above and can be effective in treating stress disorders caused from the imbalance in the nervous system.

A yeast extract according to the present invention is found to contain 18 kinds of amino acids with a great amount of aspartic acid and glutamic acid, which will be described later in Example 4. Aspartic acid and glutamic acid have a negatively charged R group. In other words, the result means that the yeast extract containing a large amount of negatively charged amino acids, which are essential to synthesis muscarinic receptors in the body, is a good source for the biosynthesis of the neurotransmitter acetylcholine distributed in the nervous system, including the parasympathetic part. Since the yeast extract according to the present invention accelerates transmission of the neurotransmitter at the parasympathetic part when the sympathetic part is excessively stimulated due to stress, the yeast extract according to the present invention has an activity as an anti-stress agent. Furthermore, the yeast extract according to the present invention can effectively relax premenstrual syndrome (PMS) and menstrual pains, which are similar to the symptoms from stress.

Food-grade yeast, generally recognized as safe (GRAS), contains 50% or greater quality protein, a large amount of minerals, and vitamin B complex. The yeast is rich in peptides as a source for the synthesis of neurotransmitters or their receptors, as described above. In particular, vitamin B and minerals contained in yeast are involved in general energy metabolism and activating the brain and neurons. Vitamin B6 (pyridoxin) is an essential nutrient for synthesizing neurotransmitters, including serotonin, dopamine, norepinephrine, gamma-amino butyric acid (GABA), and taurine. A deficiency of vitamine B6 causes dysthesia, anxiety, reduction in concentration, and hypomnesis. A deficiency of Vitamin B1 (thiamine) causes Korsakoff's syndromes such as weakness of memory, anaesthesia, and dementia. Vitamin B2 (riboflavin) acts as a coenzyme for the brain growth to be considered neurologically as an important neurotrophic factor.

In women with PMS or incapacitating menstrual pains, signs of progesterone deficiency, estrogen/progesterone imbalance, salt/water retention, prostaglandin deficiency or excess, prolactine excess, vitamin B6 (pyridoxin) deficiency, hypoglycemia, serotonin deficiency, etc. are observed. However, since the yeast extract according to the present invention contains a variety of amino acids, excess B vitamins, including B6, and excess minerals such as calcium and magnesium, hormone balance and metabolism activation are accelerated as those components are absorbed into the body, thereby rapidly reduce PMS and menstrual pains.

Furthermore, yeast contains comparatively excess selenium, which is essential to grow cranial nerve cells and to generate antioxidases. Deficiency of selenium results in depression and anxiety due to encephalopathy. Mineral enriched yeast, as an easily absorbable mineral supplement in the form of organically bound, chelated with minerals, such as selenium and chromium, is disclosed (U.S. Pat. No. 4,530,846 issued on Jul. 23, 1985, entitled “Method for the Production of Selenium Yeast”). In view of the description above, yeast is considered to be applicable as a bioactive food material and natural peptide source in the form of yeast extract or yeast-derived peptides with an activity as a good brain-neurotrophic factor capable of relaxing stress, anxiety, excitation, somnipathy due to stress, and fatigue.

In the present invention, a variety of excess exoenzymes produced are secreted through the cell wall of the yeast. Therefore, the exoenzymes are low molecular weight proteins, which are enough small to pass the yeast cell wall. Therefore, the exoenzymes can act as a brain-neurotrophic factor capable of easily passing through the brain blood barrier in the human body.

When the growth conditions, such as temperature, pH, and nutrient composition and amount, are extremely undesirable, yeast is autolysed by its own enzyme to hydrolyze the cytoplasm and subsequently destruct the cell wall so that the hydrolyzed cytoplasm comes out of the cell. Due to the yeast autolysis, bioavailability of the yeast can be enhanced. By autolysing yeast after generation of excess low molecular weight anti-stress substances in the yeast, yeast-derived peptides containing a variety of proteins, minerals, and B vitamins, which are extracted from the yeast by the autolysis and act as effective neurotrophic factors, can be obtained.

The effect of yeast extract in relaxing stress and fatigue as a neurotrophic factor has been described above. Hereinafter, the effect of relaxing PMS and menstrual pains will be described in greater detail.

First, the yeast extract contains excess choline as a complex with vitamin B. Choline is a precursor of the relaxant neurotransmitter acetylcholine. Therefore, muscular tension and pain due to uterine contraction during menstruation can be alleviated by increasing choline intake.

Choline exists in a variety of forms. Among many types of choline, salicylic choline has a pain alleviating effect similar to aspirin and thus may be effective in alleviating menstrual pains. Although the cause of menstrual pains has not been identified accurately, it is expected due to excess secretion of prostaglandin during menstruation, which is observed in women with menstrual pains. Analgesia such as acetylsalycilic acid (aspirin) or ibuprofen inhibits the synthesis of prostaglandin to reduce prostaglandin secretion. As a result, neurotransmission of the pain is inhibited to be less painful.

Second, vitamin B6 in the yeast extract composition is very important in the synthesis and metabolism of amino acids and proteins, functions as a cofactor for the synthesis of red blood cells and antibodies, and is involved in the synthesis of a variety of neurotransmitters. Deficiency of vitamin B6 results in anemia, dermatitis, neuropathy, and cramp. Also, vitamin B6 is known to be essential to relieve PMS and menstrual pains.

Third, niacin in the yeast extract composition is involved in the enzymatic reaction for the synthesis of coenzymes, NAD (nicotinamide adenine dinucleotide) and NADP (nicotinamide adenine dinucleotide phosphate). These coenzymes are known to accelerate energy generation in the intracellular mitochondria and to activate the function of brain cells to treat schizophrenia. Accordingly, niacin can be effectively used to alleviate the psychological syndromes of PMS.

Niacin is one of metabolites of the essential amino acid tryptophan. Tryptophan is a precursor of serotonin widely used to relieve PMS and menstrual pains. Accordingly, the yeast extract containing an excess of tryptophan and niacin is obviously effective in relaxing PMS and menstrual pains. The presence of excess niacin structurally similar to serotonin, which is a kind of neurotransmitter lo synthesized in the body, indicates high likelihood of converting into serotonin and thus optimizes the autoregulation mechanism in the body to treat pain, chronic fatigue, and depression.

Fourth, thiamin in the yeast extract composition is involved in the nervous system, for example, for normal heart and nerve tissue activities. Low blood thiamin content results in depression, emotional instability, somnipathy, irritability, hyperaction, etc. However, these symptoms are known to disappear by the administration of thiamin (Professor Rus Harrel, Colombia University in New York). Therefore, PMS in women, such as depression, emotional instability, somnipathy, and irritability, can be treated with the thiamin-rich yeast extract.

Fifth, riboflavin in the yeast extraction composition is involved in the synthesis of coenzymes, FMN (flavin mononucleotide) and FAD (flavin adenine dinucleotide). These coenzymes help oxidize nutrients for energy production and synthesize red blood cells and adrenocortical hormone. Accordingly, riboflavin is essential in the period of menstrual bleeding.

Sixth, the mineral selenium in the yeast extract has an antioxidant effect about 1,900 times greater than vitamin E. Neurotransmitters, including serotonin, synthesized in the body may lose their activity by oxidation in the gastrointestical tract or during transmission. Here, the antioxidant selenium aids the neurotransmitters, such as serotonin, to maximize their effects by preventing oxidation of the neurotransmitters.

Finally, an excess of minerals such as calcium and magnesium are required to relieve menstrual pains. Minerals are rapidly absorbed into the body when taken as a food, and their absorption rate is increased with the supply of amino acids. Since the yeast extract are rich in both minerals and amino acids, the minerals can be easily absorbed into the body to effectively manage PMS and menstrual pains. Through the mechanism as described above, the yeast extract can effectively alleviate PMS and menstrual pains.

In another aspect of the present invention, there is provided a PMS and menstrual pain relaxant composition further including chitosan in addition to the yeast extract.

Chitosan is known as an active material to enhance autoimmune response, to aid in the absorption of calcium, and to regulate cholesterol levels. Calcium is an essential mineral for women, especially in the period of menstrual bleeding to relieve PMS. Chitosan is highly effective in removing foreign substances as well as helps calcium absorption, and thus it is useful in relieving PMS and menstrual pains. According to the present invention, it is preferable to use a water-soluble polymeric chitosan of a molecular weight greater than or equal to about 300,000.

Alternatively, the PMS and menstrual pain relaxant composition according to the present invention may further include a herb, such as Korean angelica root, Salviae Radix, Curcuma aromatica, Zedoariae Rhizoma, mint, liquorice, ginger, gastrodia, white atractylis, Cnidium officinale, cinnamon, and ginseng.

The above-listed herbs, such as Korean angelica root, Salviae Radix, Curcuma aromatica, liquorice, mint, ginseng, cinnamon, and ginger, are known to aid in the circulation of blood, the dispersal of extravasated blood, the alleviation of pain, the increase of appetite, the activation of metabolism, especially for woman (refer to a text of herbalogy). Congestion due to the non-smooth circulation of blood during the menstrual cycle may cause menstrual pains. The above-listed herbs can alleviate the menstrual pains.

Gastrodia, cnidium officinale, white atractylis, Zedoariae Rhizoma, etc. aids in the brain blood circulation and in the generation of neurotransmitters to clear head.

Each of the components of the PMD and menstrual pain relaxant composition is ground and mixed with the composition of about 10-90% by weight yeast extract, about 50-80% by weight chitosan, and about 5-80% by weight the herb, based on the total weight of the composition. If excess yeast extract is added, a feeling of langor may result for a patient with mild pain, but an effective reduction in pain results for a patient with severe pain. Therefore, it is preferable to adjust the amount of yeast extract added within the above range.

If excess chitosan is added, the acidity of chitosan itself may act as a stimulus in the body to cause excess tension or stress in a patient with severe menstrual pains, but no adverse effect on a normal person.

The lower limits of the yeast extract and chitosan added are determined to be at least about 10% by weight and about 5% by weight, respectively, to appropriately induce the relaxation effect of the yeast extract and the stimulation effect of the chitosan for effective metabolism in the body. The upper limits of the yeast extract and chitosan added are determined to be about 90% by weight and about 80% by weight, respectively, to appropriately induce the relaxation effect of the yeast extract and the stimulation effect of the chitosan for effective metabolism in the body.

The lower limit of the herb is determined to be at least about 5% by weight by considering its effect of dispersing the extravasated blood remaining after the menstrual cycle. An effective PMS and menstrual pain relaxant composition according to the present invention can be prepared with the addition of at least about 5% by weight the herb.

A method for preparing the yeast extract according to the present invention will be described in greater detail.

In the preparation of the yeast extract according to the present invention, yeast is additionally subjected to a stress, such as high-temperature heating, ultrasonic waves, and vibrations, to secrete stress-resistant substances. This additional step performed at a temperature higher than a lethal temperature of the yeast induces autolysis to utilize active components of the yeast biomass. Here, the yeast's ability to selectively permeate the cell wall is lost, and the cell wall is destructed by enzymes existing in the yeast, such as protease, lipase, invertase, maltase, zymase, etc. Through the autolysis, a variety of taste components, including amino acids, such as glutamic acid, and nucleic acid metabolites, such as 5′-AMP, are released (Hyung-ik Song and Jung-yeub Shin, Contemporary Fermentation Engineering, 1998, 5:189). Simultaneously, pappain as a protease is added to efficiently give activities as an anti-stress agent, an anti-fatigue agent, and a neurotrophic factor.

Temperature is one of important factors affecting the growth and survival of yeast. Most microorganisms are mesophilic. Yeast has a limited growth temperature of 20-46° C. Saccharomyces cerevisiae, mesophilic yeast, is subjected to a mild thermal shock at 37° C. to induce cell resistance to a lethal temperature of 48-55° C. The resulting thermally resistant cells produce the diose trehalose and effective thermal shock proteins of 90 kDa, 70 kDa, and 60 kDa even with a rise of only 5° C. in temperature from their optimal growth temperature of 35° C. (Michell L. Deegenaars and Kenneth Watson, Environmental Microbiology, the edition of August, 1998). This report supports the fact that yeast produces anti-stress proteins when subjected to thermal stress. Based on this fact, the inventor has additionally induced the synthesis of anti-stress substances by heating at high-temperature and by applying ultrasonic waves and vibrations in the preparations of the yeast extract and yeast-derived peptide according to the present invention. In addition, the method for preparing the yeast extract and yeast-derived peptide according to the present invention involves hydrolying the yeast protein with an addition of a protease while autolysing the yeast at a high-temperature of 35-70° C., and centrifuging the hydrolytes to separate a supernatant.

To investigate the anti-stress capability of the yeast extract and yeast-derived peptide according to the present invention as a neurotrophic factor, an immobile stress test was performed based on the Brekhman and Dardymov method. In general, a series of bioreactions occurring due to stress are initiated by the central nervous system's detection of an external stimulus. Adrenocortical hormone secretion is affected according to the external stimulus to cause changes in the weight of liver, thymus, thyroid gland, and spleen and a reduction in the number of immunocytes. Also, lactate dehydrogenase (LDH) and alkaline phosphatate (ALP) levels in blood are known to change due to an effect of corticosteroid. Changes the above-listed indices have been applied to evaluate anti-stress capability (Cristina, J., Hans, W. and Hans, M., Haematological changes during acute mental stress, Bri. J. Heanat 71:564, 1971; Conner, R. L., Vernikos-Danelis, J. and Levine, S., Stress, fighting and neuroendocrine function, Nature, 2:564, 1971; Munck, A., Guyre, P. M. and Holbrook, N.J., Physiological functions of gluococorticoids in stress and their relation to pharmacological actions, Endocrine Review, 6:25, 1984). As a result of the immobile stress test with rats based on the above anti-stress indices, where changes in the weight of organs and in the level of hematobiochemical factors were measured, the yeast extract and yeast-derived peptide according to the present invention was verified to have the anti-stress effect.

A swimming endurance test was performed to measure the anti-fatigue capability, a kind of anti-stress test, of the yeast extract and yeast-derived peptide according to the present invention. In general, stress is known to affect bipolar affective reaction, motional function, and autonomous function (Yei-wan Hwang, Psychosomatosis, pp. 17-28, 33-49, and 272, Haenglim Publishing Co., Seoul, Korea). Excess exercise, such as swimming, is considered as a stress. Therefore, the swimming endurance test performed using an experimental swimming pool, where excess swimming was forced to measure the powder of endurance, to determine an anti-fatigue effect is considered to be suitable for measuring an anti-stress effect. As a result of the test, the yeast extract and yeast-derived peptide according to the present invention was proven to have excellent anti-stress and anti-fatigue effects.

As another test to measure the anti-stress effect of the yeast extract and yeast-derived peptide according to the present invention, the effect of regulating the autonomic nervous system was determined with patients with anxiety. 200 g of the yeast hydrolyte was orally administered three times a day for 1 week, and the effect of regulating the autonomic nervous system was measured using a stress measurement device (SA-2000, Medicore, Korea). The cardiac cycle (heart rate variability) obtained through power spectral density (PDS) analysis was analyzed for each 5-minute segment to obtain a 5-minute total power. As a result of the autonomic nerve regulation test with the administration of the yeast extract of the invention, autonomic nerve regulation and the ability to regulate against stress- and pain-inducing substances were enhanced with the expectation of its effect on painful PMS similar to stress symptoms.

To verify the bioactivity of the mineral-enriched yeast extract according to the present invention greater than other peptide substances, a macrophage activity test was performed on the mineral-enriched yeast extract to measure immunity. Macrophages, immunocytes involved in both congenital and adaptive immune systems, are critical in the cell-mediated immunity (CMI) to provide antigens for inducing lymphocytes through digestion and successive decomposition and other processes of externally introduced substances, to secrete specific substances, such as immunoregulatory cytokine, and to produce nitric oxide (NO) having the function of killing foreign invaders, antigens. Macrophages are divided into inflammatory macrophages and activated macrophages depending on activity. Inflammatory macrophages are produced when exposed to inflammation inducer substances, such as thioglycolate. The inflammatory macrophages have phagocytic activity and surface adherence, and increase the secretion of prostaglandin, the ability to synthesize protein of a variety of enzymes, such as plasminogen activating enzyme, elastase, collagenase, etc., cell size, and the release of a number of cellular discharges.

Macrophages activated by cytokines, such as IFN-γ and TNF, and lipopolysaccaride (LPS) from gram-negative microorganisms have anti-cancer and anti-microbial effects. In the cytotoxic mechanism of the macrophages with respect to cancer, cytokines, such as TNF-α, IL-1, IL-6, IL-8, and IL-12, hydrogen peroxide (H₂O₂), nitric acid (NO), and cytolytic protease which are released from the activated macrophages are known to have a toxicity with respect to cancer cells. The macrophages activated by such lectins, large molecular proteoglycan, and polysaccharide primarily suppress oncogenesis and then tumor metastasis, and have the ability to distinguish oncocytes and normal cells. Although a target structure the macrophage can detect has not been identified yet, unlike the phagocytosis mechanism of the macrophage, the macrophage adheres to a target oncocyte to release lysosomal enzymes before lysis of the target oncocyte. Meanwhile, non-activated macrophages have a weak toxicity to tumor cells and thus needs to be activated for enhanced oncolytic abilities. This fact supports that immunotherapy to lead macrophage activation can be an effective therapy. Accordingly, the microphage activity was tested for the mineral-enriched yeast extract according to the present invention. As a result, it was proven that the mineral-enriched yeast extract according to the present invention is effective in enhancing immunity.

The bioactivity of the mineral-enriched yeast extract according to the present invention greater than other peptide substances was additionally verified through an intestinal immunity activity test to measure immunity. Immunocytes constitute tissues or organs, called the lymphatic system, for effective immune reactions. The lymphatic lo system is classified into primary (or central) lymphoid organs, including bone marrow and thymus, for producing lymphocytes, and secondary lymphoid organs, including lymph nodes, spleen, and mucosa-associated lymphoide tissues (MALT), for providing conditions or environments for the contact of the lymphocytes and antigens and the interactions between lymphocytes. The MALT, which is located at a region susceptible to microorganisms, plays an important role in regional, mucoca immune reactions, so it is called the mucosal immune system. The MALT, which induces a defective reaction against antigens entering the body via ingestion or inspiration, is classified into the gut-associated lymphoid tissues (GALT) located in digestive track, the bronchous-associated lymphoid tissues (BALT) located in the musculi canal, and the nasal-associated lymphoid tissues (NALT) located in the junction of the plate and nose. Among these types of lymphoid tissues, the GALT as the largest lymphoid issue present in the intestinal mucosa is especially significant in the body protective system. The alimentary mucosa having a large surface area is always exposed to a number of different microorganisms and plant-derived heterologous proteins or compounds, which supports the immunological significance of the GALT.

Peyer's patches used in the examples according to the present invention to be described later, which have the typical structure of the GALT, are an aggregate of nodi lymphatici mesenterici distributed in the small intestinal mucosa. Peyer's patches are easily observed, and most are observed in the ileum. Activated lymphocytes in the Peyer's patch release a variety of cytokines, including IL-6 and GM-CSF (granulocyte macrophage-colony stimulating factor), to regulate immune or inflammatory reactions by regulating the growth, migration, proliferation of bone marrow cells, white blood cells, and hematogenic cells. Based on this fact, in an example according to the present invention to be described later the degree of proliferation of bone marrow cells by cytokines secreted with the activation of the Peyer's patch cells using the mineral-enriched yeast extract according to the present invention was measured to determine enhanced immunoactivity.

In general, a stimulus to the human body is known to be injurious if it's intensity is over the limit, thereby causing tension headaches, migraine headaches, hypertension, indigestion, fatigue, or generalized headaches. If such a stimulus is prolonged chronically, non-specific general adaptative syndromes, such as neuropathies or gastropathies, may result (Yei-wan Hwang, Psychosomatosis, pp. 17-28, 33-49, and 272, Haenglim Publishing Co., Seoul). As described above, the yeast extract and yeast-derived peptide according to the present invention have an anti-stress effect and can be used as a tranquilizer or a relaxtant. In particular, it is greatly expected to use the yeast extract and yeast-derived peptide according to the present invention as a sleeping aid for nervous people with sleeping problems. The yeast extract and yeast-derived peptide according to the present invention are useful as a source for auxiliary health foods and special nutritional foods having an activity as a brain-neurotrophic factor for relieving the above symptoms.

As described above, the yeast extract and yeast-derived peptide according to the present invention are effective in the regulation of the autonomic nervous system, for example, in alleviating stress and inducing deep sleep. The yeast extract and yeast-derived peptide according to the present prevent invention are believed to be an effective neurotrophin for normalizing the neurological function without any side effect for excess doses.

Unlike conventional side-effect inducing therapeutic drugs for nervous and stress symptoms, such as a tranquilizer, an anti-anxiety agent, a sleeping drug, etc., the yeast extract and yeast-derived peptide according to the present invention prepared from a natural source and having anti-stress and neurotrophic effects can effectively regulate the autonomic nervous system to alleviate a number of stress symptoms without any side effect. In particular, the yeast extract and yeast-derived peptide according to the present invention are applicable as a tranquilizer, an anti-stress agent, a sleeping aid, an anti-fatigue agent, and PMS and menstrual pain relaxants.

The yeast extract and yeast-derived peptide according to the present invention having the anti-stress and neurotrophic effects are available as a substitute for conventional side-effect inducing psychotrophic drugs in the preparation of medicines, active foods, medicines and feed for animals, etc. For use of the yeast extract and yeast-derived peptide according to the present invention as therapeutic agents, a variety of known pharmaceutical methods can be applied in the preparation of those drugs. The yeast extract and yeast-derive peptide according to the present invention may be processed alone or mixed with a pharmaceutically safe carrier, vehicle, diluent, etc. into powder, granule, tablet, capsule, or injection form to be orally or non-orally administered.

When the yeast extract and yeast-derived peptide according to the present invention are used as a therapeutic agent, its dose can be appropriately determined depending on the age, sex, state, and symptom of a patient. Preferably, the yeast extract or yeast-derived peptide according to the present invention is used at a dose of about 500 mg a day for adults. For a patient with insomnia, a capsule containing 250 mg of the yeast extract or yeast-derived peptide powder is administered twice a day.

For use of the yeast extract according to the present invention as a PMS and menstrual pain relaxant, the yeast extract is ground, and capsules are filled with the yeast extract powder. Alternatively, the yeast extract powder may be processed into tablets. When a dose of 1-2 capsules (or tablets) two or three times a day, each capsule (or tablet) containing 180 mg of the yeast extract powder, is taken with excess water, PMS, menstrual pains, hysterorrhea, and dysmenorrhea in females are effectively alleviated.

The yeast extract and yeast-derived peptide according to the present invention or a composition containing the same are applicable in the preparation of heath beverages effective in relieving females of PMS, menstrual pains, hysterorrhea, etc.

### EXAMPLES

The present invention now will be described more full with reference to the accompanying drawings, in which preferred examples of the invention are shown. This invention may, however, be embodied in different forms and should not be constructed as being limited to the examples set fourth herein; rather, these examples are provided so that this disclosure will be thorough and complete, and will fully convey the concept of the invention to those skilled in the art.

### Example 1

Saccaromyces cerevisiae (raw yeast purchased from Jenico Co.), food-grade yeast strain, was incubated in a YM medium at 24° C. for 48 hours with the supply of oxygen.

The resulting cultures were centrifuged at 15,000 rpm for 15 minutes to remove a supernatant. The remaining yeast precipitate was washed twice and diluted with 10-fold (v/v) sterile water. The yeast was autolyzed at a high temperature of 50° C. by an autolytic enzyme which the yeast inherently has, and simultaneously yeast protein was hydrolyzed into peptides at a initial pH of 4.0 for 48 hours with an addition of 1% protease (papain 30,000) for accelerating the hydrolysis.

The resulting hydrolytes were centrifuged at 15,000 rpm to separate a supernatant as a yeast extract according to the present invention. The yeast extract was freeze-dried and labeled with “Sample 1” to be used in the following examples.

### Example 2

1) Screening of Enhanced Yeast Strain.

Saccaromyces cerevisiae strain 7904, recognized as food-grade and obtained from the Korean Collection for Type Culture (KCTC), was incubated for 48 hours under stress from continuous 45 kHz-ultrasonic waves and vibrations resulting from a waterfall generated by a water-pump, to screen a surviving yeast strain resistant again the stress. The surviving yeast strain was plated on YM agar plate (containing 3 g/L yeast extract, 3 g/L malt extract, 5 g/L peptone, 10 g/L glucose, and 15 g/L agar) to screen an enhanced yeast strain forming a largest colony at a highest growth rate.

2) Incubation

The screened enhanced yeast strain was placed on YM medium and incubated in a fermentor supplied with oxygen, at 24° C., for 48 hours. The resulting cultures were centrifuged at 15,000 rpm for 15 minutes to remove a supernatant. The remaining yeast paste precipitate was diluted with 10-fold (v/v) 1% peptone buffer. In a fermentor equipped with an ultrasonic wave generator and a vibrator, the yeast paste was subjected to stress from heating at a high temperature of 35-45° C. and 45 kHz-ultrasonic waves for 30 second. This application of the stress was repeated at a 5-min interval for a total of 8 hours while another stress of vibrations from a waterfall generated by a water-pump was applied to the yeast paste, to secrete excess anti-stress substances.

3) Autolysis and Hydrolysis

The cultures from the incubation process was autolyzed at a high temperature of 50° C. by an autolytic enzyme which the yeast inherently has, and simultaneously yeast protein was hydrolyzed into peptides at a pH of 4.0 for 48 hours with an addition of 1% protease (papain 30,000) for accelerating the hydrolysis.

After the hydrolysis, the degree of hydrolysis was calculated by dividing a protein concentration of the supernatant by a total protein concentration of the yeast. To determine the protein concentration, the autolyzed yeast paste was centrifuged to recover a supernatant. After dilution of the supernatant, 500 μL of the dilute was pipetted into a tube and mixed with the same portion of a protein-quantitative reagent. The mixture was reacted at 60° C. for 60 minutes with stirring and measured at 562 nm using a spectrophotometer. As a result, the degree of hydrolysis was 55%.

4) Separation, Purification, and Drying of Hydrolytes

The resulting hydrolytes from the autolysis and hydrolysis was centrifuged at 15,000 rpm to recover an aqueous supernatant. The recovered supernatant was labeled “Sample 2”, whereas the precipitate from the centrifugation was labeled “Sample 3” to be used in the following examples. The supernatant and the precipitate were freeze-dried and ground to produce yeast extract powder and precipitate powder, respectiv ly.

### Example 3

A yeast extract was prepared in the same manner as in Example 2. The resulting yeast extract was subjected to separation and purification using a ultrafiltration membrane having a molecular weight cutoff (MWCO) value of 10,000, followed by freeze-drying, to produce natural yeast-derived peptides having a molecular weight smaller than or equal to 10,000.

The molecular weight distribution of the yeast peptides was measured by gel filtration chromatography. In gel filtration chromatography, the molecular weight (MW) of a target molecule is relatively measured based on the detection time of a standard molecule whose molecular weight has been known. As an example, if a molecule of a MW of 10,000 has a peak at 40.107 min, a molecule having a peak after 43 min can be estimated to be smaller than or equal to a MW of 10,000.

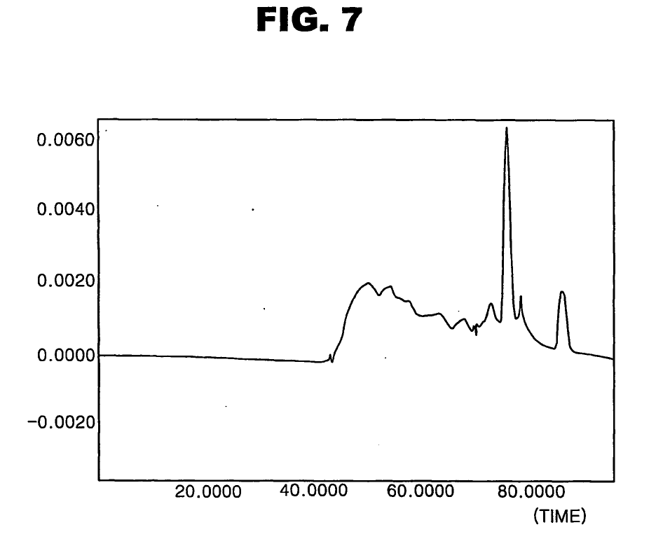

The result of the molecular weight measurement for the yeast-derived peptides prepared in this example is shown in FIG. 7. In FIG. 7, most peaks appear after 40 min. Therefore, the yeast-derived peptides prepared in this example are considered to be smaller than or equal to 10,000 daltons in molecular weight.

5% solution of the yeast-derived peptides was filtrated using a ultrafiltration membrane (having an MWCO of 5,000) to separate peptides smaller than 5,000 daltons from the larger peptides of 5,000 daltons or greater. As a result, 85% of the total yeast-derive peptides had a molecular weight smaller than 5,000 daltons.

### Example 4

Composition Analysis

The general components of the yeast hydrolytes prepared in Example 1 were analyzed by AOAC methods: moisture content by an air oven drying method at 105° C., crude protein by a microKjeldahl method, crude lipid by Soxhlet extraction, and crude ash by ashing at 550° C.

The results are shown in Table 1. As shown in Table 1, protein content was highest as 60.1 %, and carbohydrate was comparatively high as 26.9%. Comparing to general yeast extract containing 45-60% protein and 35-45% carbohydrates, the yeast hydrolytes according to the present invention was slightly higher in protein content and slightly lower in carbohydrate content than the general yeast extract.

TABLE 1

| | | Feature of Yeast Extract |
|---|---|---|
| Analyzed Item | Method | (Present Invention) |
| Appearance | Sensory test | Yellowish brown powder |
| Moisture | Air Oven Dry Method | 4.7% (max. 7%) |
| Crude lipid | Soxhlet Extraction Method | 0.3% (max. 7%) |
| Crude protein | Semi-micro-Kejeldahl | 60.1% (min. 30%) |
| | method (nitrogen | |
| | coefficient = 6.25) | |
| Carbohydrate | — | 26.9% (max. 30%) |
| Sodium | I.C.P. method | 721.2 (mg/100 g, max. 750) |
| Vitamin B1 | — | 26.12 (mg/100 g, min. 20) |
| Vitamin B2 | — | 20.13 (mg/100 g, min. 15) |
| Vitamin B6 | — | 10.61 (mg/100 g, min. 5) |
| Vitamin B12 | — | 135.8 (mg/100 g, min. 100) |
| E. coli. | — | Negative |
| UV stability | 12-hour irradiation (30% | Mild yellow (no change in |
| | yeast extract solution) | color) |
| Acid resistance | 0.67% | No precipitate (after 12- |
| | (equivalent to citric acid) | hour left at room |
| | (30% SCP-20 solution) | temperature) |
| Thermal | heating in 100° C. water for | No significant change in |
| resistance | 30 min (30% SCP-20 | color |
| | solution) | |

Amino acid contents in protein were analyzed by the following method. 10 g of the yeast extract obtained from Example 2 was dehydrated with cooling acetone and dried on a filter paper in a dry oven at 60° C. 5 mg of the dried sample was placed into a hard test tube and degassed with an addition of 5 mL of 6N HCl, followed by tight sealing. After hydrolysis at 110° C. for 24 hours, hydrolytes were washed with a small amount of distilled water 2-3 times and concentrated and dried at 50° C. by an evaporator to remove the HCl. The resulting concentrate was dissolved in a buffer and eluted into an amino acid analyzer (Deckman System 6300, USA) equipped with a 10 cm-ion exchange column (No. 338051). The amino acid composition of the yeast extract according to the present invention as the result of the analysis is shown in Table 2.

TABLE 2

| Amino Acid | Mol % |
|---|---|
| Glutamic acid | 14.2 |
| Leucine | 6.9 |
| Lysine | 5.2 |
| Aspartic acid | 5.0 |
| Alanine | 4.7 |
| Histidine | 4.3 |
| Isoleucine | 3.9 |
| Phenylalanine | 3.7 |
| Valine | 3.4 |
| Tyrosine | 3.5 |
| Threonine | 3.0 |
| Glycine | 3.0 |
| Serine | 2.8 |
| Arginine | 2.3 |
| Proline | 2.0 |
| Tryptophan | 1.4 |
| Methionine | 1.5 |
| Cysteine | 0.8 |

As shown in Table 2, the yeast-derived peptide prepared according to the present invention contained 18 kinds of different amino acids with high aspartic acid and glutamic acid contents. As is apparent from the result of the amino acid analysis, the variety of amino acids and peptides can act as anti-stress neurortrophic factors for regulating imbalance in the autonomic nervous system caused under excess stress.

### Example 5

The freeze-dried powder of the supernatant (Sample 2) prepared in Example 2 was used. Korean angelica root, Salviae Radix, Curcuma aromatica, Zedoariae Rhizoma, mint, liquorice, and ginseng were ground and mixed in the same weight ratio to prepare a herbal mixture. Water-soluble polymeric chitosan (from Jakwang Chitosan Co.) of a molecular weight greater than 300,000 daltons was purchased.

The yeast extract, the chisosan, and the herbal mixture were mixed in a weight ratio of 40:30:30, and each capsule was filled with 180 mg of the mixture.

### Example 6

Preparation of Beverage

1) Filtration and Purification

The supernatant from Example 2 was mixed with active carbon (5% by weight of the supernatant), and stirred with about 20-fold water. The mixture was sterilized in a pressure sterilizer at 121° C. for 15 minutes, followed twice by filtration with suction to produce a decolorized, deodorized, purified yeast extract.

2) Preparation of Beverage Composition

The yeast extract prepared through the filtration and purification was diluted with 70 weight part of water. 7% by weight liquid fructose, 4% by weight glucose, 5.9% by weight ume juice concentrate were added into the dilute to prepare a beverage composition.

3) Effect of Additives

To compensate for the inherent poor preference of the source material yeast, a sweetener and an acidulant were added in the preparation of an anti-stress beverage with the yeast extract. Sensory evaluation was performed by a 5-scale test to determine the effect of elevating the preference and an optimal mixing ratio. On the 5-scale test, each sensory characteristic was evaluated using five levels, including end and middle levels, extremely dislike (score 1), moderate (score 3), extremely like (score 5). The result of the sensory evaluation is shown in Table 3.

TABLE 1

| | | | | Overall | |
|---|---|---|---|---|---|
| Additive | Color | Flavor | Taste | Preference | Remarks |
| Sweetener | | | | | |
| 3% Liquid | 4.2 | 3.8 | 2.5 | 3.5 | 5% Ume juice |
| fructose | | | | | concentrate |
| 5% Liquid | 4.2 | 3.9 | 3.0 | 3.7 | added |
| fructose | | | | | |
| 7% Liquid | 4.4 | 4.1 | 3.5 | 4.0 | |
| fructose | | | | | |
| 9% Liquid | 4.2 | 4.1 | 3.5 | 3.9 | |
| fructose | | | | | |
| 2% Glucose | 4.0 | 3.9 | 2.5 | 3.5 | |
| 4% Glucose | 4.0 | 3.9 | 3.5 | 3.8 | |
| 6% Glucose | 4.0 | 3.9 | 3.2 | 3.7 | |
| Acidulant | | | | | |
| 4% Ume juice | 3.5 | 3.4 | 3.9 | 3.6 | 8% Liquid |
| concentrate | | | | | fructose |
| 6% Ume juice | 4.0 | 4.0 | 4.3 | 4.1 | added |
| concentrate | | | | | |
| 8% Ume juice | 4.1 | 4.2 | 4.0 | 4.1 | |
| concentrate | | | | | |

The effect of additive was investigated with different kinds of additives at different mixing ratios. As shown in Table 3, the overall preference was best when 7% liquid fructose was added together with 5% ume juice concentrate which was previously determined to be optimal. When glucose was added as a sweetener, the overall preference was best at 4%. There was no difference in preference for color and flavor between different glucose levels, but there was for taste. When the ume juice concentrate was added in different amounts together with 8% liquid fructose, the overall preference was the same, between 6% and 8%, but flavor preference was greatly different between the two levels. In other words, the addition of liquid fructose or glucose was effective in the improvement of preference for taste, and the addition of ume juice concentrate provided a cool feeling by enhancing the flavor and a sour taste. Based on the results of the sensory evaluation, proper amounts of liquid fructose and glucose added as a sweetener were determined to be about 7% and 4%, respectively, and an proper amount of ume juice concentrate added as an acidulant was determined to be in the range of about 6-8%.

### Experimental Example

The yeast extract according to the present invention was tested in the following examples for its activities as an anti-stress agent, an anti-fatigue agent, an autonomic nerve regulator, and a PMS and menstrual pain relaxant.

### Experimental Example 1

Anti-stress Activity Test (1) (by Ph.D. Kyung-mi Kim, Life Science Institute at Korea University)

For an anti-stress activity test, five-week old, about 180 g male sprague-dawley rats (from Biolink Co.) were adapted for 1 week in a room at a temperature of 18-23° C. through 12-hour illumination (from 7 a.m. to 7 p.m.) each day. The animals received free access to both feed (solid feed from Cheilijedang Co.) and tap water throughout the experiment. Three kinds of samples were used: “Sample 1” prepared through yeast hydrolysis at a high temperature of 50° C. according to the general yeast extract preparation method as in Example 1, “Sample 2” of the supernatant from the centrifugation in Example 2 after yeast hydrolysis following the application of ultrasonic waves and vibrations to induce stress, and “Sample 3” of the precipitate from the centrifugation of Example 2. All of the three samples were freeze-dried and ground for the experiment.

The male sprague-dawley rats were orally administered with the samples in distilled water at a dose of 1 g per body weight in kilograms, once a day for eight consecutive days; three rats for each sample. After a 6-day lapse from the administration, each of the rats was moved into a cylindrical can of a 5-cm-width and a 12-cm-length fixed at an angle of 45° to induce stress for 48 hours.

After 3 hours from the final administration, the rats were anesthetized with ether, and the thymus, spleen, kidneys, and thyroid gland were removed. The weights of the organs were measured and compared with untreated control groups which was subjected to stress. The results are shown in Table 4.

TABLE 4

| | Spleen | Kidneys | | |
|---|---|---|---|---|
| | (mg/100 g of | (mg/100 g of | Thymus | |
| Sample | body weight) | body weight) | (mg/100 g of body weight) | |
| Control Group | 279.28 ± 10.24 | 876.62 ± 19.79 | 313.89 ± 21.69 | |
| (No Stress) | | | | |
| Control Group (Stress) | 216.13 ± 6.77* | 1043.83 ± 55.31 | 221.14 ± 21.53 | 5.36 ± 0.47* |
| Sample 1 | 234.11 ± 5.31^{b} | 945.18 ± 9.95^{a} | 236.51 ± 29.29 | 7.06 ± 0.59^{a} |
| Sample 2 | 243.82 ± 16.28^{a} | 922.26 ± 11.93^{a} | 251.43 ± 3.83 | 5.71 ± 0.34 |
| Sample 3 | 226.43 ± 1.84^{b} | 1020.18 ± 44.04 | 211.41 ± 10.30 | |

*, *Significantly different from no-stress control group at p < 0.05 and p < 0.01, respectively.
^{a,b}Significantly different from stressed control group at p < 0.1 and p < 0.05, respectively.

As shown in Table 4, great changes in the weight of the organs associated with the production of stress hormones and the immune system for the stressed control group were observed for the stressed control group; the weights of the spleen, and thymus were reduced, and the weight of the kidneys was increased. In contrast, for the rats administered with Sample 1 and Sample 2, reductions in the weights of the spleen, and thymus due to stress were significantly suppressed, and an increase in the weight of the kidneys due to stress is significantly suppressed. Accordingly, both of the yeast hydrolytes (Sample 1) prepared by the general method and the yeast hydrolytes (Sample 2) subjected to stress from the ultrasonic waves and vibrations had an effective anti-stress activity. From this result, it is believed that the high-temperature yeast hydrolysis and autolysis processes themselves may induce stress, as when the ultrasonic waves or vibrations are intentionally applied.

### Experimental Example 2

Anti-stress Activity Test (2) (by Ph.D. Kyung-mi Kim, Life Science Institute at Korea University)

Stress is known to increase the size of the immune organs, including the spleen, to reduce the number of lympocytes, and to alter blood enzyme level, for example, lactate dehydrogenase (LDH) in blood. Also, an increase in blood corticosteroid level due to stress affects lipid metabolism, thereby elevating blood cholesterol level. Based on this fact, the activities of LDH, glutamate oxaloacetate transaminase (GOT), glutamate pyruvate transaminase (GPT), and alkaline phosphatase (ALP) and total cholesterol level in serum were measured to verify the samples according to the present invention for anti-stress effects. The results are shown in Table 5.

TABLE 5

| Sample | Serum GOT | Serum GPT | Serum Total Cholesterol | | Serum ALP |
|---|---|---|---|---|---|
| Control Group | 124.18 ± 2.64 | 40.62 ± 1.99 | 81.03 ± 6.08 | 538.07 ± 66.39 | 48.97 ± 8.05 |
| (No Stress) | | | | | |
| Control Group | 203.6 ± 59.70** | 58.14 ± 3.51 | 154.32 ± 9.73** | | 12.57 ± 1.29** |
| (Stress) | | | | | |
| Sample 1 | 182.24 ± 21.73 | 43.10 ± 5.09 | 125.0 ± 81.51^{a} | 718.40 ± 59.18^{a} | 15.15 ± 1.08 |
| Sample 2 | 141.63 ± 3.79^{a} | 31.50 ± 2.07 | 100.59 ± 1.56^{b} | 562.40 ± 58.02^{a} | 17.10 ± 0.90^{a} |

| | | | | |
|---|---|---|---|---|
| Sample 3 | 212.69 ± 10.61 | 55.37 ± 3.37 | 129.04 ± 20.87 | 19.10 ± 1.00^{a} |

*,**Significantly different from no-stress control group at p < 0.05 and p < 0.01, respectively.
^{a},^{b}Significantly different from stressed control group at p < 0.05 and p < 0.01, respectively.

As shown in Table 5, serum and total cholesterol levels were elevated by the application of stress whereas increases in those levels were significantly reduced by the administration of the samples according to the present invention. Also, the activities of the serum transaminases were elevated by the application of the stress, but were significantly lowered, especially for GOT level, by the administrations of Sample 1 and Sample 2 as compared to the stressed control group. Serum ALP activity was significantly lowered by the stress, but that reduction was significantly suppressed by the administration of Samples 1, 2, and 3.

These results support that Samples 1 and 2 according to the present invention show high anti-stress activities, especially Sample 2.

### Experimental Example 2

Anti-fatigue Activity Test (by Ph.D. Kyung-mi Kim, Life Science Institute at Korea University)

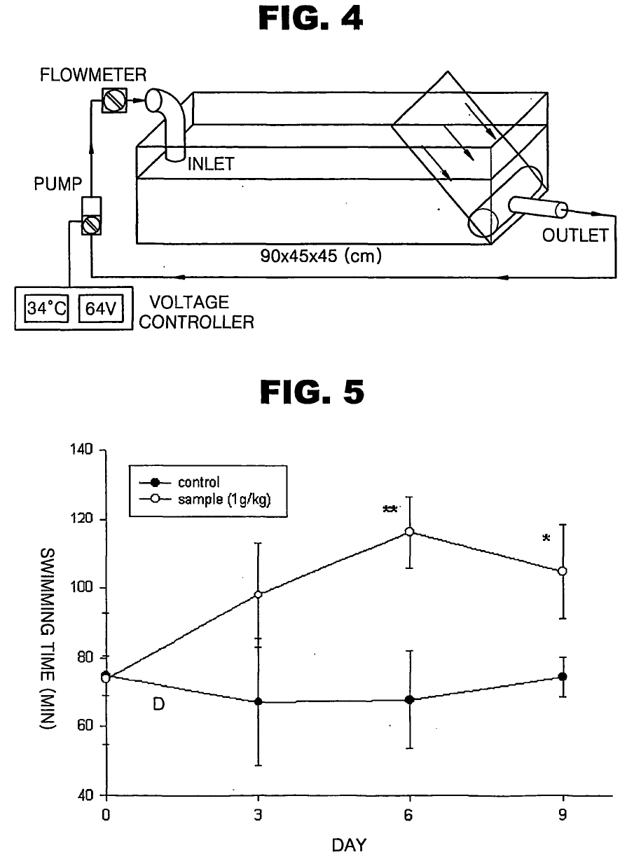

As another measure of the anti-stress activity of the yeast extract according to the present invention, the anti-fatigue activity of the yeast extract was determined by measuring swimming endurance, which is believed to be related to anti-stress effects. A swimming endurance test was conducted using an experimental swimming pool (Matsumoto et al, 1996), as shown in FIG. 4. Seven-week old, about 128 g male ICR mice (from Korean International Experimental Animal Center) were adapted for 1 week, three per cage (33×23×12 cm) in a room at a temperature of 22-24° C. and 50% humidity through 12-hour illumination (from 7 a.m. to 7 p.m.) each day. The animals received free access to both feed (solid feed from Cheilijedang Co.) and tap water throughout the experiment. A control group of mice was orally administered with the feed alone at a dose of 1 g per kilogram of body weight, and an experimental group of mice was orally administered with the yeast extract at a dose of 1 g per kilogram of body weight prepared by yeast hydrolysis at a high temperature of 50° C., as in Example 1. The swimming endurance test was performed three times over 9 days. The number of mice in each of the control and experimental groups was six. For the swimming endurance test, the acrylic plastic swimming pool (90×45×45 cm) shown in FIG. 4 was filled with water to a 35 cm-depth and maintained at 34° C. and a water flow rate of 8L/min. Here, the flow of water was induced by controlling the voltage of a pump using a voltage controller and maintained constant using a flowmeter (Type F45500, Blue White Co., Westmister, Calif., USA).

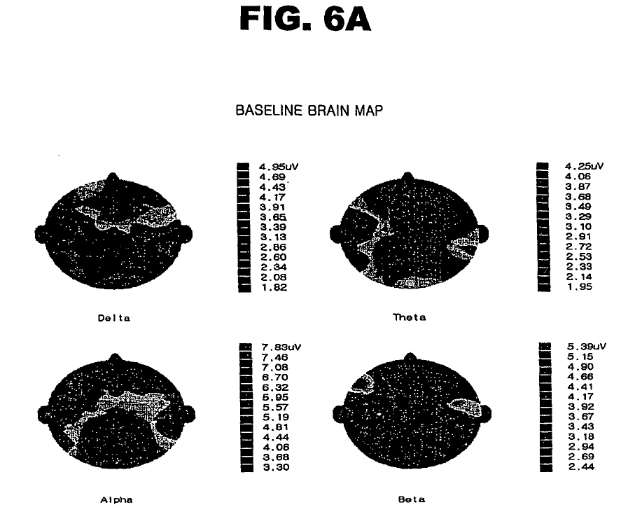

To minimized deviations in the physical activities of the mice and experimental data, the swimming endurance test was performed in the period of time from 1 p.m. to 5 p.m. The limit of swimming time of the mice was counted from the point of time after a 7-second lapse from observing the mice sink into the water. The swimming time was determined to be the period of time from the start of swimming to that time limit (Matsumoto et al., 1966). The results are shown in FIG. 5.

As shown in FIG. 5, the swimming time was markedly prolonged for the experimental group orally administered with the yeast extract according to the present invention, compared to the control group. This result verifies the yeast extract according to the present invention has anti-fatigue and anti-stress activities.

### Experimental Example 3

Autonomic Nerve Regulation Activity Test

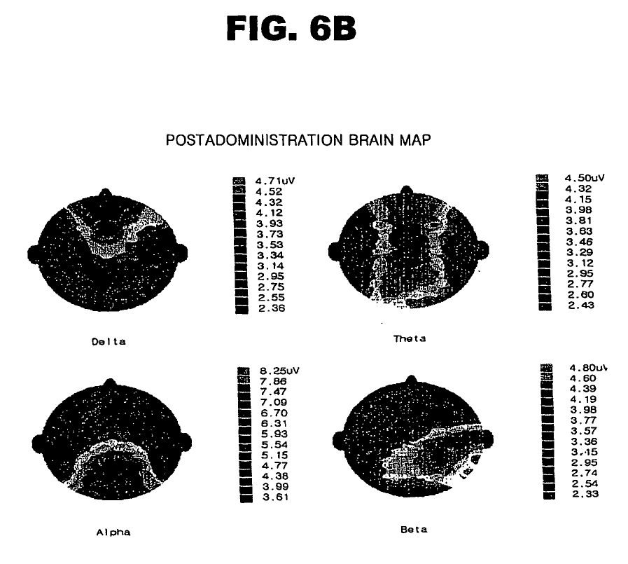

The yeast extract prepared in Example 1 was given to patients with anxiety for an autonomic nerve regulation activity test. In general, stress endurance is closely associated with the flexibility of the sympathetic nerve and parasympathetic nerve. Accordingly, heart rate variability is measured for a significant index of stress endurance, TP. The index of TP means the total power over the very low frequency (VLF), low frequency (LF), and high frequency (HF) bands during a 5-minute heart rate variability measurement, and reflects the overall activity of the autonomic nervous system, including the sympathetic nerve directly affected by stress. The result of this measurement is shown in FIG. 6. The autonomic nerve regulation activity was apparent in the experimental group treated with the yeast extract of the invention whereas no great difference in TP in the control group given a placebo. This increased activity in the autonomic nerve regulation supports that the ability to endure in stress and the ability to regulate other pain-inducing substances can be enhanced by the yeast extract according to the present invention. Therefore, it is believed that the yeast extract according to the present invention would be effective in alleviating PMS causing stress and pains.

### Experimental Example 4

Bone Marrow Cell Proliferating Activity through Peyer's Patch

The activity was measured in accordance with the procedure of Hong et al. ¹⁵) Yeast hydrolysate of present invention from Saccharomyces cerevisiae was administered orally into C3H/HeJ mice (Daehan Biolink Co., Korea) at different doses, and the mice received distilled water alone as the control. After the oral administration for 7 consecutive days, suspensions of Peyer's patch cells in RPMI 1640 medium supplemented with 5% FBS (RPMI 1640-FBS) were prepared from the small intestine of C3H/HeJ mice. Two hundred μm of aliquots of the cell suspension (2×10 ⁶ cells/ml) were cultured for 5 days at 37° C. in a humidified atmosphere of 5% CO₂-95% air. The resulting culture supernatant (50 μI) was incubated with bone marrow cell suspension (2.5×10⁵ cells/ml) from untreated C3H/HeJ mice for 6 days in the same incubator. After 20 μl of Alamar Blue™ solution was added and the cells were then continuously cultured for 5-24 hours, the fluorescence intensity was measured to count cell numbers by Spectrafluor Plus (Tecan, Austria) at an excitation wavelength of 544 nm and an emission wav length of 590 nm during cultivation.

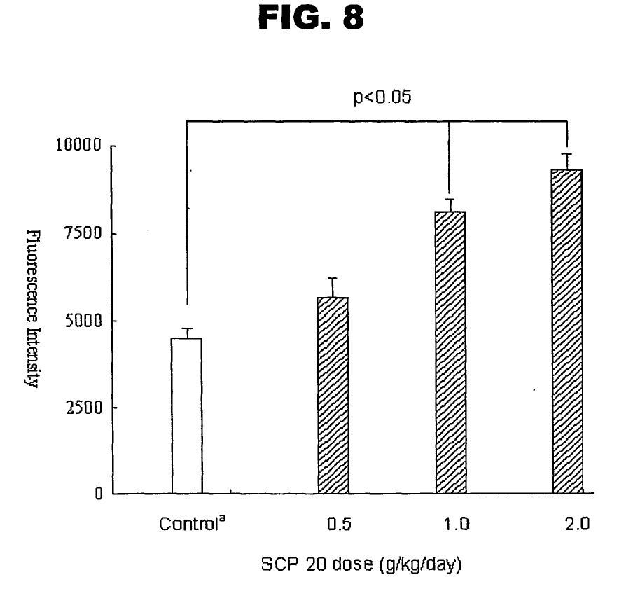

Bone marrow cells were proliferated in a dose-dependent manner and reached almost plateau over 2.0 g/kg per day, as shown in FIG. 8. When 2.0 g/kg per day of yeast hydrolyte of this invention was used for stimulation of Peyer's patch cells, the number of bone marrow cells increased up to 2.1-fold measured by Alamar Blue™ reduction assay (FIG. 8). This observation suggests that several kinds of growth factors may contribute to the proliferative response. Lymphocytes, such as typically activated T cells, are known to secrete growth factors such as IL-6,²⁴) and these growth factors stimulate proliferation of hematopoietic cells and follow by differentiation to granulocytes or macrophages. In order to know whether yeast hydrolyte of this invention enhances IL-6 secretion from Peyer's patch cells, Peyer's patch cells of C3H/HeJ administered the yeast hydrolyte 7 days at different doses were cultured for 5 days, and then levels of IL-6 were examined. The IL-6 content increased in the conditioned medium significantly when Peyer's patch cells were administered with the yeast hydrolyte (at 2.0 g/kg per day, 2.3-fold) (Table 6). These results suggest that IL-6 may contribute to the proliferation of bone marrow cells in a part. Because IL-6 is a multifunctional hematopoietic growth factor, they stimulate the granulopoiesis.

TABLE 6
Effect of Orally Administered Hydrolyte from
Saccharomyces cerevisiae on IL-6 in the Culture
Supernatant of Peyer's Patch Cells and Macrophages

| | Dose | Relative content of IL-6 (%) | |
|---|---|---|---|
| Sample | (g/kg per day) | Peyer's patch^{b} | Macrophage^{c} |
| Control^{a} | — | 100 ± 3.4 | 100 ± 4.3 |
| Yeast hydrolyte | 0.5 | 122 ± 2.5* | 119 ± 3.8* |
| | 1.0 | 188 ± 5.7* | 165 ± 10.6 |
| | 2.0 | 231 ± 9.7* | 187 ± 8.6* |

^{a}Saline alone was administered for control.
^{b}Peyer's patch cells were obtained from C3H/HeJ mice (n = 4) fed SCP-20 at different doses for 7 days, and pooled and cultured at a density of 4 × 10⁵ cells/well for 5 days in vitro. The resulting cell-free supernatants were subjected to ELISA for IL-6.
^{c}Macrophage cells were obtained from ICR mice (n = 4) fed SCP-20 at different doses for 7 days, and pooled and cultured at a density of 2 × 10⁵ cells/well for 2 days in vitro. The resulting cell-free supernatants were subjected to ELISA for IL-6.
*Significant difference between control and samples at p < 0.05. Data were expressed as percent of control of mean ± S.D. of quadruplicate assays.

### Experimental Example 5

Macrophage-stimulating Activity and the Production of IL-6

1) Macrophage-stimulating Activity

Male ICR mice (from Daehan Biolink Co., Chungcheongbuk-Do, Korea), which had been orally administered with yeast hydrolyte of the invention at different doses, are injected aseptically with 1 ml of 3% thioglycollate broth via i. p. Peritoneal exudates cells were harvested by the injection of 5 ml of the cold RPMI-1640 medium (Gibco, Grand Island, N.Y.) containing 5 mM HEPES, penicillin (100 U/ml) and streptomycin (100 μg/ml). After the cells suspension was adjusted to 1×10⁶ cells/ml and incubated at 37° C. in a humidified chamber (48 h), the macrophage-stimulating activity was measured using an assay system of the cellular lysosomal enzyme based on the activity of acid phosphatase from macrophages (Bio-Rad, Model 3550-UV).

2) Determination of IL6 in Supernatant of Macrophage and Peyer's Patch Cell Cultures

The ELISA (enzyme-linked immunosorbent assay) employing the multiple antibody sandwich principle was used. After 2 μg of purified anti-mouse IL6 mAb (monoclonal antibody) (clone MP5-20F3, PharMingen, San Diego, Calif.) in 50 μl of bicarbonate buffer (pH 8.5) was adhered to each well of 96 plates, unbound antibody was removed by washing 4 times with PBS containing 0.05% Tween 20 (PBS-Tween). Samples were added to the antibody-coated wells at a 100 μl, and each 100 μl of biotinylated anti-mouse IL-6 mAb (MP5-32C11, PharMingen) in PBS containing 10% FBS was added to the same wells. After the plates were washed 6 times with PBS-Tween, alkaline phosphatase-labelled streptoavidin (Gibco, Grand Island, N.Y.) was added to each well. Each well was incubated with 150 μl of chromogenic substrate solution (1 mg of p-nitrophenyl disodium salt in 1 ml of 10% diethanolamine buffer, pH 9.8), and subsequently the absorbance at 405 nm was measured.

Statistical analysis: All results were expressed as the mean±S.D. The difference between the controls and the treatments in these experiments was tested for statistical significance by Student's t-test. A value of p<0.05 was considered to indicate statistical significance.

3) Result

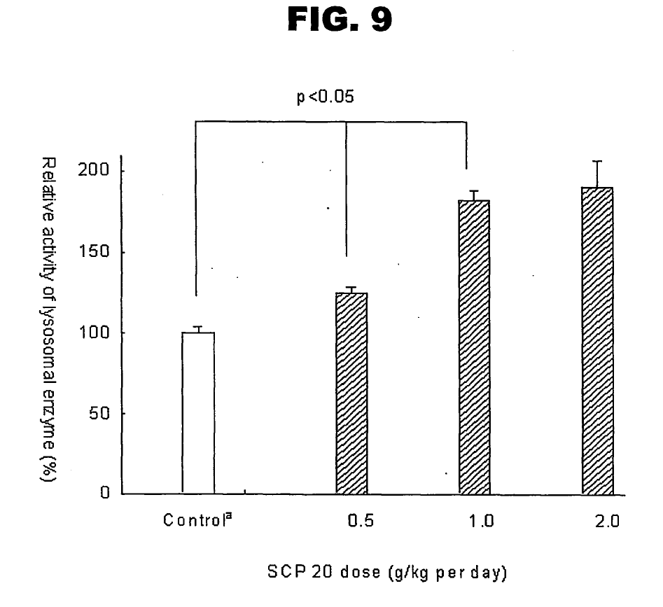

Administration of 0.5, 1.0 and 2.0 g/kg per day of yeast hydrolyte of the invention for 7 days revealed a dose-dependent increase in the relative activity of a macrophage lysosomal enzyme, as shown in FIG. 9. A significant increase in the relative activity was seen at 0.5 g/kg per day (1.3-fold of saline control), and maximum stimulation was made by 2.0 g/kg per day (1.9-fold) (FIG. 9). These results suggest that the oral administration of yeast hydrolyte of the invention enhance the stimulatory responses of macrophages. In addition, the effects of the orally administered yeast hydrolyte of the invention at different doses on IL-6 secretion, which enhances IL-2 production from T cell and stimulates proliferation of hematopoietic cells, from macrophages were investigated in mice. Oral administration of the yeast hydrolyte of the invention was found to increase significantly and dose-dependently, compared to the control, and the yeast hydrolyte of the invention stimulated the most IL-6 production at 2.0 g/kg per day (1.9-fold) (Table 6).

For activation of a macrophage function, at least one signal must be provided.²⁷) The signal, which sensitizes the macrophage to respond to the other signal, is delivered by the macrophage stimulating cytokine IFN-γ.²⁸) IFN-γ is the most specific cytokine produced by Th1, Tc and natural killer cells. It does not induce macrophage cytokine production but it regulates macrophage cytokine production, which is enhancing the production of IL-1, IL-6 and TNF-α. The cytokine network of macrophage plays an important role in the inflammatory and immune responses, and especially, IL-6 is significant in the differentiation and as growth factor of macrophage.

From this experiment, it is believed that oral administration of the yeast hydrolyte of the invention hydrolyzed from Saccharomyces cerevisiae may modulate IL-6 production in macrophage. Enhancement in the production of the cytokine, IL-6 by the oral administration of the yeast hydrolyte of the invention suggests that the yeast hydrolyte of the invention might induce the activation of macrophage. In addition, oral administration of the yeast hydrolyte of the invention is believed to enhance secretion of hematopoietic growth factors from Peyer's patch cells. Since Peyer's patch cells are mainly composed of T and B cells, and T cells are known as a source of CSFs and various bytokines as well as macrophages, T cell activation, which is caused by oral administration of the yeast hydrolyte of the invention, may contribute to secretion of hematopoietic growth factors such as IL-6 from Peyer's patch cells. Especially, since the cytokines such as IL-6 is important in the systemic immunocytes, the orally administered yeast hydrolyte of the invention would regulate the systemic immune system according to the Peyer's patch-mediated mechanism.

Therefore, studies on fractionation and purification-activity relationship of these active substances, yeast hydrolyte of the invention, on biological activity will give useful important information.

### Experimental Example 6

Clinical Test (by neuropsychiatrist Won-jun Hwang)

To verify the anti-stress effect of the present invention in the human body, a clinical test was performed on three groups of patients with neuropsychiatric problems: a group with insomnia, a group with anxiety, and a group with headaches.

Baseline brain mapping was performed on those groups of patients after a 3-day postadministration (Feb. 6, 2001). After 3-day administration of the yeast extract according to the present invention at a dose of 500 mg a day, the brain mapping was conducted (Feb. 9, 2001). The results are shown in FIGS. 6A and 6B.

As shown in FIGS. 6A and 6B, as a result of the baseline brain mapping, an asymmetric increase in theta wave activity was observed in almost all the brain region. This increased theta wave activity means a psychic tension state due to increased mental activity. In contrast, after the administration of the yeast extract according to the present invention for 3 days, a symmetrical distribution of the theta wave in the central and parietal lobes was observed. This pattern of theta wave distribution appears in a psychologically stable state such as when one is completely relaxed or in a hypnotic or hypnagogic stage.

### Experimental Example 7

Clinical Test (by neuropsychiatrist Won-jun Hwang)

Capsules prepared in Example 5 were orally administered 2-3 times a day, 1-2 capsules each, given to thirty women in 20-35 ages with PMS, menstrual pains, or hysterorrhea before their menstrual cycle or for menstrual pains during the menstrual cycle.

The subjects were asked for thirty questions about changes after the administration, ten relating to physical symptoms, ten relating to emotional symptoms, and ten relating to behavior symptoms. The thirty questions were:

A) Physical symptoms

1) Asthenia or fatigue

2) Increased or decreased appetite

3) Breast fullness or tenderness

4) Headaches

5) Nausea or vomiting

6) Dizziness or giddiness

7) Swelling

8) Abdominal aches or unwell and intermittent abdominal cramping

9) Insomnia

10) Changes in sexual relationship or in interests

B) Emotional symptoms

1) Serious mood swings in a day

2) Be anxious or agitated

3) Be nervous or restless

4) Be sad or depressed

5) Nag or quarrel over a trivial matter

6) Be hysteric or irritable

7) Feel guilty

8) Be unappreciative or impatient with others' defects or mistakes

9) Think over or worry about discomforting matters

10) Be blunt

C) Behavioral symptoms

1) Be slow in or poor at movement

2) Often make a mistake or causes an accident (e.g., falls over, gets a cut from a knife, or breaks something by mistake)

3) Think about death or self-murder (e.g., wants to be dead and gone in sleep)

4) Be unwilling to speak or go out

5) Abuse drugs (excitant, sedative, etc.) or smoke or drink heavily

6) Be rude or behave annoyingly

7) Decreased efficiency or activity at home and office

8) Avoid social activities and want to stay at home

9) Too lazy to do domestic duties (cleaning, washing, etc.)

10) Have less leisure hours (hobbies, watching TV, reading, etc.)

The subjects were asked to answer each of the questions and grade the degree of each symptom on a 6-point scale: 1 for none, 2 for almost none, 3 for slight, 4 for moderate, 5 for fairly severe, and 6 for extremely severe. In addition, the overall feeling before and after the administration and pain relaxation time and its duration were asked. The average of those scores from the subjects for each symptom was calculated.

As a result, pain relaxation appeared within 10 minutes at the earliest and 1 hour at latest, with a duration of about 4-6 hours. PMS alleviation was significant in all of the physical, emotional, and behavioral symptoms. The average degree of each symptom before and after the administration and its variation are shown in Table 7.

TABLE 7

| Symptom | Before | After | Variation |
|---|---|---|---|
| Physical Symptoms | 32 | 23 | 9 |
| Emotional Symptoms | 30 | 24 | 6 |
| Behavioral Symptoms | 29 | 22 | 7 |
| Overall Symptoms | 91 | 69 | 22 |

It was found that the PMS and menstrual pain relaxant composition according to the present invention can effectively relieve severe pains, paralysis, indigestion, and vomiting before or during the menstrual cycle and can activate blood circulation and improve temper. Most of the female subjects answered that their pessimistic and depressed mood disappeared after taking the PMS and menstrual pain relaxant composition according to the present invention. Furthermore, the PMS and menstrual pain relaxant composition according to the present invention was known to be effective against lumbago during menstruation. A woman with serious lumbago answered that her lumbago disappeared within 30 minutes after taking 2 capsules (each containing 180 mg of the PMS and menstrual pain relaxant composition according to the present invention) in favor of future administrations. Women with the inability to fall into deep sleep or those who experience sleep interruptions due to their menstrual pains were able to sleep deeply after the administration of the PMS and menstrual pain relaxant composition according to the present invention. Unlike conventional analgesia that completely kill pains by causing paralysis and unconsciousness, the PMS and menstrual pain relaxant composition according to the present invention does not cause unconsciousness although there remains a mild but not bad pain. However, most of the female subjects preferred the PMS and menstrual pain relaxant composition according to the present invention to conventional analgesia. Also, the effect of warming of the abdomen and extremities was observed in most of the female subjects. The PMS and menstrual pain relaxant composition according to the present invention was more effective in women who took the composition before menstruation. For women who took the PMS and menstrual pain relaxant composition according to the present invention after pains had occurred, the pain relaxation effect appeared about 3-4 hours later, on average. Some of the female subjects who had endured pains without taking analgesia due to its medical side effects responded that they will take the PMS and menstrual pain relaxant composition according to the present invention, which is derived from a food source, to enhance their health and alleviate pains, instead of conventional medicinal analgesia.

### INDUSTRIAL APPLICABILITY

The present invention discloses new uses of yeast extract, which previously had solely been used as a food source, as an anti-stress agent and a neurotrophin. The yeast extract according to the present invention has applications as a substitute for conventional psychominetics that have a number of side effects and as an active food source.

Instead of using a conventional complicated process of gene-recombinant strain expression, natural neurotrophins can be easily prepared from yeast-derived peptides obtained by the hydrolysis of good-grade yeast. Neurotrophins according to the present invention are prepared such that they pass through the brain blood barrier even when orally administered. The new development of uses of the yeast extract according to the prevent invention includes its uses as a medicine source for an anti-stress agent, an anti-stress agent, and a natural neurotrophin, as well as an active food source.

Conventionally, medicines containing, for example, serotonin or melatonin, have been administered to alleviate PMS or menstrual pains with the prescription of doctors or pharmacists. However, the yeast extract or yeast-derived peptides according to the present invention are prepared from a safe food source that effects no resistance or side effects and thus can be conveniently purchased and taken without a doctors' or pharmacists' prescription to relieve normal women of their monthly PMS or menstrual pain suffering.

### Reference

1. Task Force of the European Society of Cardiology and the North American Society of Pacing and Electrophysiology. Heart rate variability standards of measurement, physiological interpretation, and clinical use. Cir. 93: 1043-1065 (1996)

2. Airaksinen, K. E., Ylitalo, A., Niemela, M. J., Tahvanainen, K. U., and Huikuri, H. V. Heart rate variability and occurrence of ventricular arrhythmias during balloon occlusion of a major coronary artery. Am. J. Cardiol. 83: 1000-1005 (1999)

3. Brekhman I. I., Dardymov I. V., Lloydia, 32, 46-51 (1969).

4. Matsumoto K., Ishihara K., Tanaka K., Inoue K., Fushiki T., J. Appl. Phys., 81, 1843-1849 (1996).

5. Hong T., Matsumoto T., Kiyohara H., Yamada H. Phytomedicine, 5, 353-360 (1998).

6. Pagé B., Pagé M., Noë l C. Int. J. Oncol., 3, 473-476 (1993).

7. Herscowitz B. H., Holden H. T., Bellanti J. A., Ghaffar A. (ed.), “Induction and Collection of Peritoneal Exudates Macrophages” Marcel Dekker Inc., New York, 1981.

8. Hirano T., Taga T., Nakano N., Yasukawa K., Kashiwamura S., Shimizu K., Makajima K., Pyun K-H., Kishimoto T., P. Natl. Acad. Sci. USA, 82, 5490-5494 (1985).

9. Wong G. G., Witek-Giannoutii J. S., Temple P. A., Kriz R., Ferenz C., Hewick R. M., Clark S. C., Ikebuchi K., Ogawa M., J. Immun., 140, 3040-3044 (1988).

10. Okano A., Suzuki C., Takatsuki F., Akiyama Y., Koike K., Ozawa K., Hirano T., Kishimoto T., Nakahara T., Asano S., Transplantation, 48, 495-498 (1989).

11. Adams D. O., Hamilton T. A., Annu Rev. Immunol., 2, 283-318 (1984).

12. Farran M. A., Schreiber R. D., Annu. Rev. Immunol., 22, 571-611 (1993).

13. Cao H., Wolff R. G., Meltzer M. S., Crawford R. M. J. Immunol., 143, 3524-3531 (1989).

14. Liles W. C., Van Voorhis W. C., J. Infect. Dis., 172, 1573-1580 (1995).

15. Mosmann T., Coffman R., Ann. Rev. Immunol., 7, 145-173 (1989).

16. Lotz M., Jirik F., Kabouridis P., Tsoukas C., Hirano T., Kishimoto T., Carson D. A., J. Exp. Med., 167, 1253-1258 (1988).

What is claimed is:

1. A yeast extract derived from yeast, the yeast extract having activities as an anti-stress agent, an anti-fatigue agent, premenstrual syndrome and menstrual pain relaxants, and a neurotrophic factor.

2. The yeast extract of claim 1, wherein the yeast is autolyzed at a temperature of about 35-70° C.

3. The yeast extract of claim 2, wherein the yeast is hydrolyzed with a protease during or after the autolysis.

4. The yeast extract of claim 3, wherein the yeast hydrolytes is centirfuged to obtain a supernatant.

5. A yeast-derived peptide having molecular weight of smaller than or equal to about 10,000 daltons obtained by ultrafiltring the supernatant of claim 4.

6. The yeast-derived peptide of claim 5, comprising glutamic acid and aspartic acid of about 15-25 mol % each.

7. The yeast-derived peptide of claim 5 capable of passing through a brain blood barrier.

8. A method for preparing a yeast extract having activities as an anti-stress agent, an anti-fatigue agent, premenstrual syndrome and menstrual pain relaxants, and a neurotrophic factor, the method comprising:
incubating a strain of yeast until a maximum growth phase;
inducing autolysis of the yeast at a temperature of about 35-70° C. and simultaneously hydrolyzing the yeast with an addition of a protease; and
attaining a supernatant by centrifuging hydrolytes generated in the hydrolysis.

9. The method of claim 8, further comprising screening a new strain of the yeast survived resisting to stress by heating at high-temperature, applying ultrasonic waves and vibrations, and changing pH to a degree not to cause destruction before the incubation until the maximum proliferation stage.

10. The method of claim 8, further comprising inducing secretion of metabolites resistant to stress by applying a physical or chemical stress selected from the group consisting of high-temperature heating, ultrasonic waves, vibrations, and pH variations, to a degree not to cause destruction before the autolysis.

11. A method for preparing a yeast-derived peptide having activities as an anti-stress agent, an anti-fatigue agent, premenstrual syndrome and menstrual pain relaxants, and a neurotrophic factor, the method comprising attaining peptides having a molecular weight of about less than or equal to 10,000 daltons by ultrafiltrating the supernatant from the centrifugation in the method of any of claims 8 through 10.

12. A method for preparing an active beverage having activities as an anti-stress agent, an anti-fatigue agent, premenstrual syndrome and menstrual pain relaxants, and a neurotrophic factor, the method comprising:
mixing the supernatant from the centrifugation in the method of any of claims 8 through 10 with active carbon in water and sterilizing the mixture under pressure; and
purifying the sterilized mixture until it loses its color by filtering the sterilized mixture with suction.

13. An anti-stress agent comprising the yeast extract of any of claims 1 through 4 or the yeast-derived peptide of any of claims 5 through 7 as an active component.

14. The anti-stress agent of claim 13 having an activity as a tranquilizer, a relaxant, an anti-anxiety agent, or a sleeping aid.

15. An anti-fatigue agent comprising the yeast extract of any of claims 1 through 4 or the yeast-derived peptide of any of claims 5 through 7 as an active component.

16. A neurotrophin comprising the yeast extract of any of claims 1 through 4 or the yeast-derived peptide of any of claims 5 through 7 as an active component.

17. A premenstrual syndrome and menstrual pain relaxant composition comprising the yeast extract of any of claims 1 through 4 or the yeast-derived peptide of any of claims 5 through 7 as an active component.

18. The premenstrual syndrome and menstrual pain relaxant composition of claim 17 comprising:
about 10-90% by weight dried powder of the yeast extract or the yeast-derived peptide;
about 5-80% by weight chitosan; and
about 5-80% by weight herbal powder of 5-80% by weight, based on the total weight of the premenstrual syndrome and menstrual pain relaxant composition.

19. The premenstrual syndrome and menstrual pain relaxant composition of claim 18, wherein the citosan is a water-soluble macromolecule of a molecular weight greater than or equal to about 300,000.

20. The premenstrual syndrome and menstrual pain relaxant composition of claim 18, wherein the herbal powder is derived from at least one selected from the group consisting of Korean angelica root, Salviae Radix, Curcuma aromatica, Zedoariae Rhizoma,mint, liquorice, ginger, gastrodia, white atractylis, cnidium officinale, cinnamon, and ginseng.

21. An active beverage having activities as an anti-stress agent, an anti-fatigue agent, premenstrual syndrome and menstrual pain relaxants, and a neurotrophic factor, the active beverage comprising the yeast extract of any of claims 1 through 4 or the yeast-derived peptide of any of claims 5 through 7.

22. The active beverage of claim 21 comprising:
about 0.1-10% by weight the yeast extract or the yeast-derived peptide;
about 10-25% by weight common additives for beverage including a sweeter and an acidulant, based on the total weight of the bioactive beverage; and
the balance water.
